(19) United States
(12) Patent Application Publication
(10) Pub. No.: US 2026/0217726 A1
(43) Pub. Date: Jul. 30, 2026

(54) IMPROVED SYNTHESIS OF BUPRENORPHINE, ANALOGUES AND INTERMEDIATES

(71) Applicant: RIVER STONE BIOTECH APS, Copenhagen (DK)

(72) Inventors: Aaron GREGSON, Carina (AU); Peter John SCAMMELLS, North Balwyn (AU)

(21) Appl. No.: 19/146,914

(22) PCT Filed: Feb. 8, 2024
(86) PCT No.: PCT/EP2024/053120

§ 371 Date: Jul. 9, 2025

(30) Foreign Application Priority Data
Feb. 8, 2023 (EP) 23155490.8

(51) Int. Cl. C07D 489/12

(52) U.S. Cl. CPC C07D 489/12

(57) ABSTRACT

The present invention relates to methods for preparing buprenorphine, analogues, and intermediates. More particularly, the present invention relates to an improved synthesis of a key intermediate toward buprenorphine.

### TECHNICAL FIELD

The present invention relates to methods for preparing buprenorphine, analogues, and intermediates. More particularly, the present invention relates to an improved synthesis of a key intermediate toward buprenorphine.

### BACKGROUND

Total synthesis of natural opiate compounds or semisynthetic opioids is complex and not commercially competitive (Rinner et al., Top. Curr. Chem. 309:33-66 (2012)). While natural opiates are obtained from plants, opioids are often obtained semi-synthetically using natural opiate precursors. Buprenorphine and other semisynthetic opioids are, or can be, made from thebaine, an opiate alkaloid (Hudlicky, Can. J. Chem. 93 (5): 492-501 (2015)). Thebaine is currently obtained by crop cultivation of and extraction from plants of the Papaver genus. Several possible methods to prepare buprenorphine have been reported from thebaine. A known and likely commercial route to buprenorphine is made up of 6 major steps, starting from thebaine. (Machara et al., Adv. Synth. Catal. 354 (4): 613-26 (2012); Werner et al., J. Org. Chem. 76 (11): 4628-34 (2011)). The first 3 steps are a Diels-Alder reaction of thebaine with methyl vinyl ketone to form a 4+2 product, hydrogenation of the carbon-carbon double bond of the resultant product, and addition of a tertiary butyl group via a Grignard reaction. The final steps are N- and O-demethylation and cyclopropyl alkylation. The number of steps can increase to 8, if the N- and O-demethylation and N-alkylation steps are performed in 2 stages, rather than 1. The order of the hydrogenation and Grignard steps may be reversed but most, if not all, economically viable preparations include the 3 above-mentioned steps prior to the N-demethylation step. One challenge of this known preparation of buprenorphine is the exchange of the N-methyl group for an N-cyclopropyl group. N-demethylation methods can involve highly toxic reagents such as cyanogen bromide (von Braun, J. Chem. Ber., 33:1438-1452 (1900)) and chloroformate reagents (Cooley et al., Synthesis, 1:1-7 (1989); Olofson et al., J. Org. Chem., 49:2081-2082 (1984)). Attempts to reduce impurities and improve yields have been made by avoiding the O-demethylation step, by using oripavine as starting material, but a principal obstacle to an efficient synthesis remains the N-demethylation step. While prior work such as WO 2021/144362 demonstrates the synthesis of buprenorphine starting from nororipavine, there remains a need for a further improved route to buprenorphine and analogues thereof, such as a route that is more efficient due to e.g. improved yield, decreased impurities and/or produces less waste using reagents and solvents suitable for industrial scale.

### SUMMARY

Over this background art the present inventors have now developed reaction conditions that enable production of the Grignard adduct, Pg-O-IIIA-Pg*, such as BnO-IIIA-Bn in high yields with few impurities. In particular, the selected reaction conditions are suitable for process chemistry and scale-up, rendering the method applicable for an industrial setting.

Surprisingly, the reaction proceeds in high yield and with very few impurities when conducted at room temperature, or at temperatures such as from 15° C. to 60° C., in particular from 20° C. to 30° C. Being able to conduct the reaction on larger scale without the need for cooling to 0° C. or lower provides a significant operational benefit.

Further, the present inventors have developed improved conditions for isolating the Grignard adduct. These improved processes enable secure and reproducible large-scale production of complex pharmaceutical ingredients, such as buprenorphine.

Accordingly, in a first aspect the disclosure describes a method of preparing a compound of formula Pg-O-IIIA-Pg*,

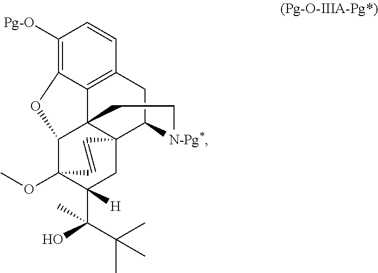

comprising mixing a compound of formula Pg-O-II-Pg*,

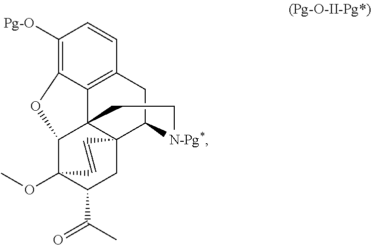

with a tert-butylmagnesium compound and one or more additives in a solvent system to provide the compound of formula Pg-O-IIIA-Pg*; wherein each Pg and Pg* is a hydroxy-protecting group and Pg and Pg* the same or different.

In a further aspect, the present disclosure describes a method of preparing a compound of formula Pg-O-IIIA-Pg*,

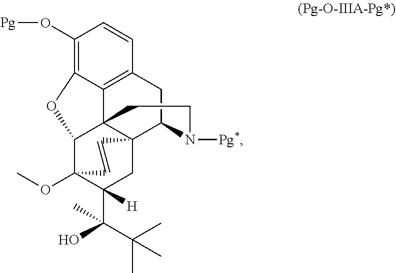

comprising mixing a compound of formula Pg-O-II-Pg*,

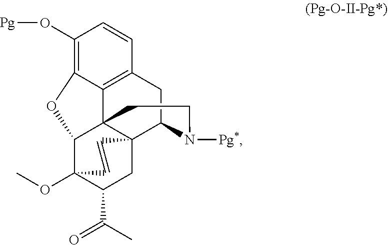

with 4 to 8 equivalents of a tert-butylmagnesium compound and 3 to 6 equivalents of diglyme in a solvent system comprising cyclopentyl methyl ether (CPME) to provide the compound of formula Pg-O-IIIA-Pg*; wherein each Pg and Pg* is a hydroxy-protecting group and Pg and Pg* the same or different; wherein each Pg and Pg* is selected from the group consisting of: benzyl (Bn), methoxymethyl (MOM), tetrahydropyranyl (THP), tert-butyl (tBu), allyl, tert-butyldimethylsilyl (TBDMS), tert-butyldiphenylsilyl (TBDPS), acetyl (Ac), pivaloyl (Piv), and benzoyl (Bz).

In a further aspect, a method is provided for isolating a compound of formula Pg-O-IIIA-Pg* from a reaction mixture in a reaction container, the method comprising:
a. adding an alcoholic solvent, such as ethanol to the reaction mixture;
b. heating the reaction mixture to at least 60° C., for example to the boiling point of the alcoholic solvent, for a predefined amount of time;
c. cooling the reaction mixture to from 40 to 55° C., optionally over the course of at least 30 minutes;
d. further cooling the reaction mixture to from 0 to 30° C. and stirring the mixture for a predefined amount of time thereby obtaining a solid;
e. adding further of the alcoholic solvent to the reaction mixture; and
f. collecting the solid by filtration and optionally drying the solid in vacuo.

In a further aspect, the disclosure describes a method for preparing buprenorphine, or a salt thereof, comprising the method as defined herein.

### INCORPORATION BY REFERENCE

All publications, patents, and patent applications referred to herein are incorporated by reference to the same extent as if each individual publication, patent, or patent application was specifically and individually indicated to be incorporated by reference. In the event of a conflict between a term herein and a term in an incorporated reference, the term herein prevails and controls.

### DETAILED DESCRIPTION

The features and advantages of the present invention is readily apparent to a person skilled in the art by the below detailed description of embodiments and examples of the invention with reference to the figures and drawings included herein.

### Definitions

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

All methods described herein can be performed in any suitable order of steps unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

As will be understood by one of ordinary skill in the art, each embodiment disclosed herein can comprise, consist essentially of or consist of its particular stated element, step, ingredient or component.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range.

All percentages, ratios and proportions herein are by weight, unless otherwise specified. A weight percent (weight %, also as wt. %) of a component, unless specifically stated to the contrary, is based on the total weight of the composition in which the component is included (e.g., on the total amount of the reaction mixture).

Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Several embodiments of this invention are described herein. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All patents and printed publications are individually incorporated herein by reference in their entirety.

Terms used herein may be preceded and/or followed by a single dash, “-”, or a double dash, “=”, to indicate the bond order of the bond between the named substituent and its parent moiety; a single dash indicates a single bond and a double dash indicates a double bond or a pair of single bonds in the case of a spiro-substituent. In the absence of a single or double dash it is understood that a single bond is formed between the substituent and its parent moiety; further, substituents are intended to be read “left to right” with reference to the chemical structure referred to unless a dash indicates otherwise. For example, arylalkyl, arylalkyl-, and -alkylaryl indicate the same functionality.

For simplicity, chemical moieties are defined and referred to throughout primarily as univalent chemical moieties (e.g., alkyl, aryl, etc.). Nevertheless, such terms are also used to convey corresponding multivalent moieties under the appropriate structural circumstances clear to those skilled in the art. For example, while an “alkyl” moiety can refer to a monovalent radical (e.g. CH₃—CH₂—), in some circumstances a bivalent linking moiety can be “alkyl,” in which case those skilled in the art will understand the alkyl to be a divalent radical (e.g., —CH₂—CH₂—), which is equivalent to the term “alkylene.” (Similarly, in circumstances in which a divalent moiety is required and is stated as being “aryl,” those skilled in the art will understand that the term “aryl” refers to the corresponding divalent moiety, arylene). All atoms are understood to have their normal number of valences for bond formation (i.e., 4 for carbon, 3 for N, 2 for O, and 2, 4, or 6 for S, depending on the oxidation state of the S). Nitrogens in the presently disclosed compounds can be hypervalent, e.g., an N-oxide or tetrasubstituted ammonium salt. On occasion a moiety may be defined, for example, as —B-(A)ₐ, wherein a is 0 or 1. In such instances, when a is 0 the moiety is —B and when a is 1 the moiety is —B-A.

As used herein, the term “alkyl” or “alkane” includes a saturated hydrocarbon having a designed number of carbon atoms, such as 1 to 40 carbons (i.e., inclusive of 1 and 40), 1 to 35 carbons, 1 to 25 carbons, 1 to 20 carbons, or 1, 2, 3, 4, 5, 6, 7, 8, 9, 10, 11, 12, 13, 14, 15, 16, 17, or 18. Alkyl groups or alkanes may be straight or branched and depending on context, may be a monovalent radical or a divalent radical (i.e., an alkylene group). For example, the moiety “—(C₁-C₆ alkyl)-O—” signifies connection of an oxygen through an alkylene bridge having from 1 to 6 carbons and C₁-C₃ alkyl represents methyl, ethyl, and propyl moieties. Examples of “alkyl” include, for example, methyl, ethyl, propyl, isopropyl, butyl, iso-, sec- and tert-butyl, pentyl, and hexyl. Examples of “alkane” include, for example, methane, ethane, propane, isopropane, butane, isobutane, pentane, hexane, heptane, and octane.

The term “alkoxy” represents an alkyl group of indicated number of carbon atoms attached to the parent molecular moiety through an oxygen bridge. Examples of “alkoxy” include, for example, methoxy, ethoxy, propoxy, and isopropoxy.

The term “alkenyl” as used herein, unsaturated hydrocarbon containing from 2 to 10 carbons (i.e., inclusive of 2 and 10), 2 to 8 carbons, 2 to 6 carbons, or 2, 3, 4, 5 or 6, unless otherwise specified, and containing at least one carbon-carbon double bond. Alkenyl group may be straight or branched and depending on context, may be a monovalent radical or a divalent radical (i.e., an alkenylene group). For example, the moiety “—(C₂-C₆ alkenyl)-O—” signifies connection of an oxygen through an alkenylene bridge having from 2 to 6 carbons. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl, and 3,7-dimethylocta-2,6-dienyl.

The term “alkynyl” as used herein, unsaturated hydrocarbon containing from 2 to 10 carbons (i.e., inclusive of 2 and 10), 2 to 8 carbons, 2 to 6 carbons, or 2, 3, 4, 5 or 6 unless otherwise specified, and containing at least one carbon-carbon triple bond. Alkynyl group may be straight or branched and depending on context, may be a monovalent radical or a divalent radical (i.e., an alkynylene group). For example, the moiety “—(C₂-C₆ alkynyl)-O—” signifies connection of an oxygen through an alkynylene bridge having from 2 to 6 carbons. Representative examples of alkynyl include, but are not limited to, acetylenyl, 1-propynyl, 2-propynyl, 3-butynyl, 2-pentynyl, and 1-butynyl.

The term “aryl” represents an aromatic ring system having a single ring (e.g., phenyl) which is optionally fused to other aromatic hydrocarbon rings or non-aromatic hydrocarbon or heterocyclic rings. “Aryl” includes ring systems having multiple condensed rings and in which at least one is carbocyclic and aromatic, (e.g., 1,2,3,4-tetrahydronaphthyl, naphthyl). Examples of aryl groups include phenyl, 1-naphthyl, 2-naphthyl, indanyl, indenyl, dihydronaphthyl, fluorenyl, tetralinyl, and 6,7,8,9-tetrahydro-5H-benzo[a]cycloheptenyl. “Aryl” also includes ring systems having a first carbocyclic, aromatic ring fused to a nonaromatic heterocycle, for example, 1H-2,3-dihydrobenzofuranyl and tetrahydroisoquinolinyl. The aryl groups herein are unsubstituted or, when specified as “optionally substituted”, can unless stated otherwise be substituted in one or more substitutable positions with various groups as indicated.

The term “heteroaryl” refers to an aromatic ring system containing at least one aromatic heteroatom selected from nitrogen, oxygen and sulfur in an aromatic ring. Most commonly, the heteroaryl groups will have 1, 2, 3, or 4 heteroatoms. The heteroaryl may be fused to one or more non-aromatic rings, for example, cycloalkyl or heterocycloalkyl rings, wherein the cycloalkyl and heterocycloalkyl rings are described herein. In one embodiment of the present compounds the heteroaryl group is bonded to the remainder of the structure through an atom in a heteroaryl group aromatic ring. In another embodiment, the heteroaryl group is bonded to the remainder of the structure through a non-aromatic ring atom. Examples of heteroaryl groups include, for example, pyridyl, pyrimidinyl, quinolinyl, benzothienyl, indolyl, indolinyl, pyridazinyl, pyrazinyl, isoindolyl, isoquinolyl, quinazolinyl, quinoxalinyl, phthalazinyl, imidazolyl, isoxazolyl, pyrazolyl, oxazolyl, thiazolyl, indolizinyl, indazolyl, benzothiazolyl, benzimidazolyl, benzofuranyl, furanyl, thienyl, pyrrolyl, oxadiazolyl, thiadiazolyl, benzo[1,4]oxazinyl, triazolyl, tetrazolyl, isothiazolyl, naphthyridinyl, isochromanyl, chromanyl, isoindolinyl, isobenzothienyl, benzoxazolyl, pyridopyridinyl, purinyl, benzodioxolyl, triazinyl, pteridinyl, benzothiazolyl, imidazopyridinyl, imidazothiazolyl, benzisoxazinyl, benzoxazinyl, benzopyranyl, benzothiopyranyl, chromonyl, chromanonyl, pyridinyl-N-oxide, isoindolinonyl, benzodioxanyl, benzoxazolinonyl, pyrrolyl N-oxide, pyrimidinyl N-oxide, pyridazinyl N-oxide, pyrazinyl N-oxide, quinolinyl N-oxide, indolyl N-oxide, indolinyl N-oxide, isoquinolyl N-oxide, quinazolinyl N-oxide, quinoxalinyl N-oxide, phthalazinyl N-oxide, imidazolyl N-oxide, isoxazolyl N-oxide, oxazolyl N-oxide, thiazolyl N-oxide, indolizinyl N-oxide, indazolyl N-oxide, benzothiazolyl N-oxide, benzimidazolyl N-oxide, pyrrolyl N-oxide, oxadiazolyl N-oxide, thiadiazolyl N-oxide, triazolyl N-oxide, tetrazolyl N-oxide, benzothiopyranyl S-oxide, benzothiopyranyl S,S-dioxide. Preferred heteroaryl groups include pyridyl, pyrimidyl, quinolinyl, indolyl, pyrrolyl, furanyl, thienyl and imidazolyl, pyrazolyl, indazolyl, thiazolyl and benzothiazolyl. In certain embodiments, each heteroaryl is selected from pyridyl, pyrimidinyl, pyridazinyl, pyrazinyl, imidazolyl, isoxazolyl, pyrazolyl, oxazolyl, thiazolyl, furanyl, thienyl, pyrrolyl, oxadiazolyl, thiadiazolyl, triazolyl, tetrazolyl, isothiazolyl, pyridinyl-N-oxide, pyrrolyl N-oxide, pyrimidinyl N-oxide, pyridazinyl N-oxide, pyrazinyl N-oxide, imidazolyl N-oxide, isoxazolyl N-oxide, oxazolyl N-oxide, thiazolyl N-oxide, pyrrolyl N-oxide, oxadiazolyl N-oxide, thiadiazolyl N-oxide, triazolyl N-oxide, and tetrazolyl N-oxide. Preferred heteroaryl groups include pyridyl, pyrimidyl, quinolinyl, indolyl, pyrrolyl, furanyl, thienyl, imidazolyl, pyrazolyl, indazolyl, thiazolyl and benzothiazolyl. The heteroaryl groups herein are unsubstituted or, when specified as “optionally substituted”, can unless stated otherwise be substituted in one or more substitutable positions with various groups, as indicated.

The term “heterocycloalkyl” refers to a non-aromatic ring or ring system containing at least one heteroatom that is preferably selected from nitrogen, oxygen and sulfur, wherein said heteroatom is in a non-aromatic ring. The heterocycloalkyl may have 1, 2, 3 or 4 heteroatoms. The heterocycloalkyl may be saturated (i.e., a heterocycloalkyl) or partially unsaturated (i.e., a heterocycloalkenyl). Heterocycloalkyl includes monocyclic groups of three to eight annular atoms as well as bicyclic and polycyclic ring systems, including bridged and fused systems, wherein each ring includes three to eight annular atoms. The heterocycloalkyl ring is optionally fused to other heterocycloalkyl rings and/or non-aromatic hydrocarbon rings. In certain embodiments, the heterocycloalkyl groups have from 3 to 7 members in a single ring. In other embodiments, heterocycloalkyl groups have 5 or 6 members in a single ring. In some embodiments, the heterocycloalkyl groups have 3, 4, 5, 6 or 7 members in a single ring. Examples of heterocycloalkyl groups include, for example, azabicyclo[2.2.2]octyl (in each case also “quinuclidinyl” or a quinuclidine derivative), azabicyclo[3.2.1]octyl, 2,5-diazabicyclo[2.2.1]heptyl, morpholinyl, thiomorpholinyl, thiomorpholinyl S-oxide, thiomorpholinyl S,S-dioxide, 2-oxazolidonyl, piperazinyl, homopiperazinyl, piperazinonyl, pyrrolidinyl, azepanyl, azetidinyl, pyrrolinyl, tetrahydropyranyl, piperidinyl, tetrahydrofuranyl, tetrahydrothienyl, 3,4-dihydroisoquinolin-2 (1H)-yl, isoindolindionyl, homopiperidinyl, homomorpholinyl, homothiomorpholinyl, homothiomorpholinyl S,S-dioxide, oxazolidinonyl, dihydropyrazolyl, dihydropyrrolyl, dihydropyrazinyl, dihydropyridinyl, dihydropyrimidinyl, dihydrofuryl, dihydropyranyl, imidazolidonyl, tetrahydrothienyl S-oxide, tetrahydrothienyl S,S-dioxide and homothiomorpholinyl S-oxide. Especially desirable heterocycloalkyl groups include morpholinyl, 3,4-dihydroisoquinolin-2 (1H)-yl, tetrahydropyranyl, piperidinyl, aza-bicyclo[2.2.2]octyl, γ-butyrolactonyl (i.e., an oxo-substituted tetrahydrofuranyl), γ-butryolactamyl (i.e., an oxo-substituted pyrrolidine), pyrrolidinyl, piperazinyl, azepanyl, azetidinyl, thiomorpholinyl, thiomorpholinyl S,S-dioxide, 2-oxazolidonyl, imidazolidonyl, isoindolindionyl, piperazinonyl. The heterocycloalkyl groups herein are unsubstituted or, when specified as “optionally substituted”, can unless stated otherwise be substituted in one or more substitutable positions with various groups, as indicated.

The term “cycloalkyl” or “cycloalkane” refers to a non-aromatic carbocyclic ring or ring system, which may be saturated (i.e., a cycloalkyl, a cycloalkane) or partially unsaturated (i.e., a cycloalkenyl). The cycloalkyl ring can be optionally fused to or otherwise attached (e.g., bridged systems) to other cycloalkyl rings. Certain examples of cycloalkyl groups or cycloalkanes present in the disclosed compounds have from 3 to 7 members in a single ring, such as having 5 or 6 members in a single ring. In some embodiments, the cycloalkyl groups have 3, 4, 5, 6 or 7 members in a single ring. Examples of cycloalkyl groups include, for example, cyclohexyl, cyclopentyl, cyclobutyl, cyclopropyl, tetrahydronaphthyl and bicyclo[2.2.1]heptane. Examples of cycloalkanes include, for example, cyclohexane, methylcyclohexane, cyclohexanone, cyclohexanol, cyclopentane, cycloheptane, and cycloctane. The cycloalkyl groups herein are unsubstituted or, when specified as “optionally substituted”, may be substituted in one or more substitutable positions with various groups, as indicated.

The term “ring system” encompasses monocycles, as well as fused and/or bridged polycycles.

The terms “halogen” or “halo” indicate fluorine, chlorine, bromine, and iodine. In certain embodiments of each and every embodiment described herein, the term “halogen” or “halo” refers to fluorine or chlorine. In certain embodiments of each and every embodiment described herein, the term “halogen” or “halo” refers to fluorine.

The term “halide” indicates fluoride, chloride, bromide, and iodide. In certain embodiments of each and every embodiment described herein, the term “halide” refers to bromide or chloride.

The term “substituted,” when used to modify a specified group or radical, means that one or more hydrogen atoms of the specified group or radical are each, independently of one another, replaced with the same or different substituent groups as defined below, unless specified otherwise.

Specific protecting groups may be used to protect reactive functionalities of a starting material or intermediate to prepare a desired product. In general, the need for such protecting groups as well as the conditions necessary to attach and remove such groups will be apparent to those skilled in the art of organic synthesis. An authoritative account describing the many alternatives to the trained practitioner are J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie”, Houben-Weyl, 4.sup.th edition, Vol. 15/1, Georg Thieme Verlag, Stuttgart 1974, in H.-D. Jakubke and H. Jescheit, “Aminosauren, Peptide, Proteine”, Verlag Chemie, Weinheim, Deerfield Beach, and Basel 1982, and/or in Jochen Lehmann, “Chemie der Kohlenhydrate: Monosaccharide and Derivate”, Georg Thieme Verlag, Stuttgart 1974. The protecting groups may be removed at a convenient subsequent stage using methods known from the art.

As used herein, the term “benzyl” (“Bn”) includes unsubstituted (i.e., (C₆H₅)—CH₂—) and substituted benzyl (i.e., benzyl substituted at the 2-, 3-, and/or 4-position with C₁-C₈ alkyl or halide). The person of ordinary skill in the art will appreciate that oxygen protecting groups include alkoxycarbonyl, acyl, acetal, ether, ester, silyl ether, alkylsulfonyl, and arylsulfonyl. Exemplary oxygen protecting groups include allyl, triphenylmethyl (trityl or Tr), benzyl, methanesulfonyl, p-toluenesulfonyl, p-methoxybenzyl (PMB), p-methoxyphenyl (PMP), methoxymethyl (MOM), p-methoxyethoxymethyl (MEM), tetrahydropyranyl (THP), ethoxyethyl (EE), methylthiomethyl (MTM), 2-methoxy-2-propyl (MOP), 2-trimethylsilylethoxymethyl (SEM), benzoate (BZ), allyl carbonate, 2.2.2-trichloroethyl carbonate (Troc), 2-trimethylsilylethyl carbonate, trimethylsilyl (TMS), triethylsilyl (TES), triisopropylsilyl (TIPS), triphenylsilyl (TPS), t-butyldimethylsilyl (TBDMS), and t-butyldiphenylsilyl (TBDPS). A variety of protecting groups for the oxygen and the synthesis thereof may be found in “Protective Groups in Organic Synthesis” by T. W. Greene and P. G. M. Wuts, John Wiley & Sons, 1999. In certain embodiments, an appropriate oxygen protecting group may be used in place of benzyl.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

### Hydroxy-Protecting Groups

The present disclosure provides protecting groups, Pg and Pg* for a hydroxy group. Such protecting groups according to the present disclosure are functional groups that are temporarily added to the hydroxy group in order to protect it from unwanted reactions during a synthesis. The most commonly used protecting groups for hydroxy groups include ethers, such as tetrahydropyranyl (THP) or benzyloxycarbonyl (Boc), and esters, such as benzyl ester (Bn) or isobutyl ester (iBu). Once the synthesis is complete, the protecting group is removed to reveal the original hydroxy group.

In one embodiment, each Pg and Pg* is selected from the group consisting of: benzyl (Bn), methoxymethyl (MOM), tetrahydropyranyl (THP), tert-butyl (tBu), allyl, tert-butyldimethylsilyl (TBDMS), tert-butyldiphenylsilyl (TBDPS), acetyl (Ac), pivaloyl (Piv), and benzoyl (Bz).

In one embodiment, Pg and Pg* are benzyl (Bn), such that the compound of formula Pg-O-IIIA-Pg* is compound BnO-IIIA-Bn,

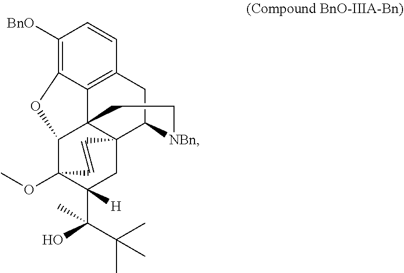 [SMILES: CO[C@]12C=C[C@@]3(C[C@@H]1[C@](C)(O)C(C)(C)C)[C@H]1Cc4ccc(OCc5ccccc5)c5c4[C@@]3(CCN1Cc1ccccc1)[C@H]2O5]

and the compound of formula Pg-O-II-Pg* is compound BnO-II-Bn,

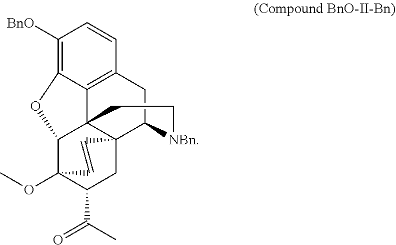 [SMILES: CO[C@]12C=C[C@@]3(C[C@@H]1C(C)=O)[C@H]1Cc4ccc(OCc5ccccc5)c5c4[C@@]3(CCN1Cc1ccccc1)[C@H]2O5]

### Reaction Conditions

In some embodiments, the present disclosure provides a method of preparing a compound of formula Pg-O-IIIA-Pg*,

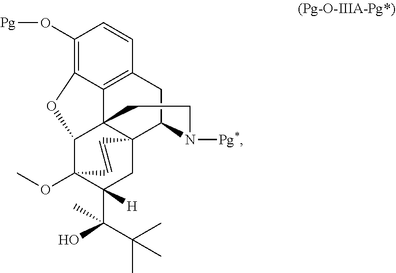

comprising mixing a compound of formula Pg-O-II-Pg*,

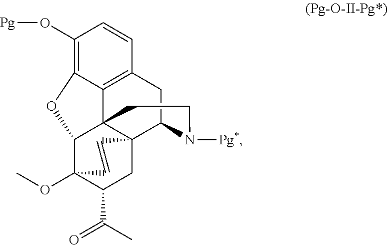

with a tert-butylmagnesium compound and one or more additives in a solvent system to provide the compound of formula Pg-O-IIIA-Pg*; wherein each Pg and Pg* is a hydroxy-protecting group and Pg and Pg* the same or different; wherein each Pg and Pg* is selected from the group consisting of: benzyl (Bn), methoxymethyl (MOM), tetrahydropyranyl (THP), tert-butyl (tBu), allyl, tert-butyldimethylsilyl (TBDMS), tert-butyldiphenylsilyl (TBDPS), acetyl (Ac), pivaloyl (Piv), and benzoyl (Bz).

In some embodiments, the present disclosure provides a method of preparing a compound of formula Pg-O-IIIA-Pg*,

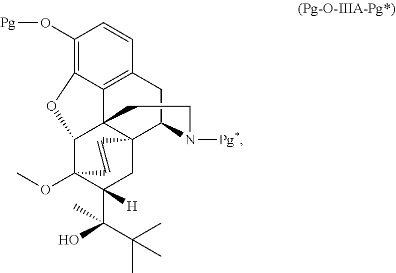

comprising mixing a compound of formula Pg-O-II-Pg*,

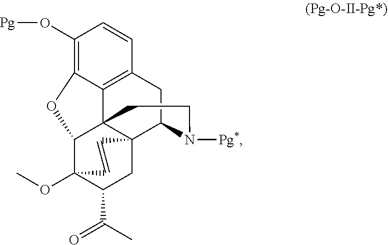

with 4 to 8 equivalents of a tert-butylmagnesium compound and 3 to 6 equivalents of diglyme in a solvent system comprising cyclopentyl methyl ether (CPME) to provide the compound of formula Pg-O-IIIA-Pg*; wherein each Pg and Pg* is a hydroxy-protecting group and Pg and Pg* the same or different; wherein each Pg and Pg* is selected from the group consisting of: benzyl (Bn), methoxymethyl (MOM), tetrahydropyranyl (THP), tert-butyl (tBu), allyl, tert-butyldimethylsilyl (TBDMS), tert-butyldiphenylsilyl (TBDPS), acetyl (Ac), pivaloyl (Piv), and benzoyl (Bz). In some embodiments, both Pg and Pg* are Bn.

### Additives

In one embodiment, the method disclosed herein comprises use of one or more additives, for example one additive. In some embodiments, the presence of the additives disclosed herein contributes to increased yield and higher purity once the starting material has reached full conversion.

In one embodiment, wherein the one or more additives are selected from the group consisting of: a non-cyclic polyether, a cyclic polyether, a Lewis acid, and a combination thereof.

In one embodiment, the non-cyclic polyether is a glyme. In one embodiment, the glyme is selected from the group consisting of: monoglyme, diglyme, triglyme, tetraglyme, ethyl glyme, ethyl diglyme, butyl glyme, butyl diglyme, and polyglyme.

In some embodiments, the glyme is diglyme.

In some embodiments, the one or more additives are added to the solvent system in a stochiometry with respect to the compound Pg-O-II-Pg* of from 1 equivalent to 8 equivalents, such as from 2 equivalents to 7 equivalents, such as from 3 equivalents to 6 equivalents, for example 4 equivalents.

### Order of Addition

In some embodiments, a solution of the Grignard i.e. the tert-butylmagnesium compound is added to a solution of the ketone, i.e. a compound of formula Pg-O-II-Pg*. In some embodiments, a solution of the ketone is added to a solution of the Grignard.

In preferred embodiments, a solution of the ketone, i.e. a compound of formula Pg-O-II-Pg* is added to the tert-butylmagnesium compound. In some embodiments, this order of addition provides an operational benefit in view of the thick mixture often resulting from complexation of the tert-butylmagnesium compound with the additive, such as diglyme.

In some embodiments, the method comprises the steps:
a. adding the tert-butylmagnesium compound to a first solvent system to provide a solution of the tert-butylmagnesium compound;
b. adding Compound Pg-O-II-Pg* to a second solvent system to provide a solution of Pg-O-II-Pg*; and
c. adding the solution of Pg-O-II-Pg* over a time period to the solution of the tert-butylmagnesium compound; or adding the solution of the tert-butylmagnesium compound over a time period to the solution of Pg-O-II-Pg*.

In some embodiments, the method comprises the steps:
a. adding the tert-butylmagnesium compound to a first solvent system to provide a solution of the tert-butylmagnesium compound;
b. adding Compound BnO-II-Bn to a second solvent system to provide a solution of Bn-O-II-Bn; and
c. adding the solution of BnO-II-Bn over a time period to the solution of the tert-butylmagnesium compound; or adding the solution of the tert-butylmagnesium compound over a time period to the solution of BnO-II-Bn.

In some embodiments, a composition or solution of the compound Pg-O-II-Pg* is prepared by reacting Compound Pg-O-I-Pg* with methyl vinyl ketone in a solvent system S2 comprising a polar protic solvent;

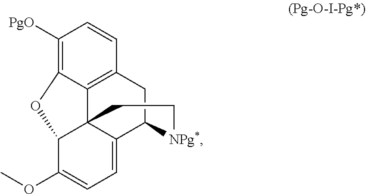

and the composition or solution of Pg-O-II-Pg* obtained is used directly in the method of preparing the compound of formula Pg-O-IIIA-Pg* disclosed herein. In some embodiments, the term “used directly” means that the solution or composition of Pg-O-II-Pg* is not purified e.g. by crystallization or other means prior to its use in the method of preparing the compound of formula Pg-O-IIIA-Pg* disclosed herein. In some embodiments, the method of the present disclosure uses a composition or solution of Pg-O-II-Pg* prepared without purification as described herein as starting material for the method of the present disclosure.

### Time

In some embodiments, the time period is from 10 to 80 minutes per mL of solution of Pg-O-II-Pg*, for example 20 minutes for 0.5 mL solution of Pg-O-II-Pg*.

In some embodiments, the time period is at least 1 minute, such as at least 2 minutes, such as at least 5 minutes, such as at least 10 minutes, such as at least 20 minutes, such as at least 40 minutes, such as at least 60 minutes, such as at least 80 minutes, such as at least 100 minutes, such as at least 120 minutes.

In some embodiments, the time period is at least 1 minute, such as at least 2 minutes, such as at least 5 minutes, such as at least 10 minutes, such as at least 20 minutes, such as at least 40 minutes, such as at least 60 minutes, such as at least 80 minutes, such as at least 100 minutes, such as at least 120 minutes.

In some embodiments, the time period is 6 hours or less, such as 5 hours or less, such as 4 hours or less, such as 3 hours or less.

In some embodiments, a compound of formula Pg-O-II-Pg*, such as BnO-II-Bn and the tert-butylmagnesium compound are mixed and allowed to react for at least 1 hour after mixing, such as from 1 hour to 24 hours after mixing.

### Temperature

In some embodiments, the method of the present disclosure is provided wherein Pg-O-II-Pg* and the tert-butylmagnesium compound are mixed at a temperature within the range of from 15° C. to 100° C., such as from 20° C. to 100° C., such as from 25° C. to 100° C., such as from 30° C. to 100° C., such as from 15° C. to 95° C., such as from 15° C. to 90° C., such as from 15° C. to 85° C., such as from 20° C. to 95° C., such as from 25° C. to 90° C.

In some embodiments, the method of the present disclosure is provided wherein BnO-II-Bn and the tert-butylmagnesium compound are mixed at a temperature within the range of from 15° C. to 100° C., such as from 20° C. to 100° C., such as from 25° C. to 100° C., such as from 30° C. to 100° C., such as from 15° C. to 95° C., such as from 15° C. to 90° C., such as from 15° C. to 85° C., such as from 20° C. to 95° C., such as from 25° C. to 90° C.

In some embodiments, Pg-O-II-Pg* and the tert-butylmagnesium compound are mixed at a temperature within the range of from 15° C. to 100° C., such as from 15° C. to 90° C., such as from 15° C. to 80° C., such as from 15° C. to 70° C., such as from 15° C. to 60° C.

In some embodiments, BnO-II-Bn and the tert-butylmagnesium compound are mixed at a temperature within the range of from 15° C. to 100° C., such as from 15° C. to 90° C., such as from 15° C. to 80° C., such as from 15° C. to 70° C., such as from 15° C. to 60° C.

### Tert-Butylmagnesium Compound

In some embodiments, the tert-butylmagnesium compound is a nucleophilic tert-butyl compound which can react with carbonyl groups to form a tertiary alcohol.

In some embodiments, the tert-butylmagnesium compound is selected from the group consisting of: a tert-butylmagnesium halide and di-tert-butylmagnesium.

In some embodiments, the tert-butylmagnesium compound is tert-butylmagnesium chloride.

In some embodiments, the tert-butylmagnesium compound is added to the solvent system in a stochiometry with respect to the compound Pg-O-II-Pg* of from 2 equivalents to 12 equivalents, such as from 2 equivalents to 11 equivalents, such as from 2 equivalents to 10 equivalents, such as from 3 equivalents to 9 equivalents, such as from 4 equivalents to 8 equivalents, such as from 5 equivalents to 9 equivalents, such as from 6 to 7 equivalents, for example 6 equivalents.

In some embodiments, the tert-butylmagnesium compound is added to the solvent system in a stochiometry with respect to the compound BnO-II-Bn of from 2 equivalents to 12 equivalents, such as from 2 equivalents to 11 equivalents, such as from 2 equivalents to 10 equivalents, such as from 3 equivalents to 9 equivalents, such as from 4 equivalents to 8 equivalents, such as from 5 equivalents to 9 equivalents, such as from 6 to 7 equivalents, for example 6 equivalents or for example 8 equivalents.

In some embodiments, the mol ratio of the tert-butylmagnesium compound is at least 6:4 and at most 12:4 with respect to the mol sum of one or more additives, optionally wherein the mol ratio is with respect to Pg-O-II-Pg* as the limiting factor, optionally wherein one additive is used, such as glyme. In some embodiments, the mol ratio of the tert-butylmagnesium compound is at least 6:4 and at most 12:4 with respect to the mol sum of one or more additives, optionally wherein the mol ratio is with respect to BnO-II-Bn as the limiting factor, optionally wherein one additive is used, such as glyme.

### Solvent Systems

In some embodiments, the solvent system, the first solvent system, and/or the second solvent system comprises a solvent having a dielectric constant of 7 or less, such as 6 or less, such as 5 or less.

In some embodiments, the solvent system, the first solvent system, and/or the second solvent system comprises a solvent having a dielectric constant of from 1 to 6, such as from 1 to 5.

In some embodiments, the solvent system, the first solvent system, and/or the second solvent system comprises an ethereal solvent.

In some embodiments, the ethereal solvent has a dielectric constant of 7 or less, such as 6 or less, such as 5 or less. In some embodiments, the ethereal solvent has a dielectric constant of from 1 to 6, such as from 1 to 5.

In some embodiments, the ethereal solvent is selected from the group consisting of: diethyl ether, cyclopentyl methyl ether (CPME), tert-butyl methyl ether (MTBE) and tetrahydrofurane (THF).

In some embodiments, the first solvent system comprises a nonpolar solvent.

In some embodiments, the nonpolar solvent of the first solvent system is toluene.

In some embodiments, the first solvent system comprises a nonpolar solvent and an ethereal solvent.

In some embodiments, the first solvent system comprises a nonpolar solvent and an ethereal solvent in a ratio of 20:80 by volume to 80:20 by volume such as 30:70, such as 40:60, such as 50:50, such as 60:40, such as 70:30, such as 80:20.

In some embodiments, the second solvent system comprises a nonpolar solvent.

In some embodiments, the nonpolar solvent of the second solvent system is toluene.

### Method of Isolation

In some embodiments, the compound Pg-O-IIIA-Pg* prepared using the method of the present disclosure is isolated using a method of isolation as disclosed herein. In some embodiments, the method of isolation is conducted essentially as demonstrated in Example 5.

In some embodiments, a method for isolating a compound of formula Pg-O-IIIA-Pg* from a reaction mixture in a reaction container is provided, the method comprising:
a) adding an alcoholic solvent, such as ethanol to the reaction mixture;
b) heating the reaction mixture to at least 60° C., for example to the boiling point of the alcoholic solvent, for a predefined amount of time;
c) cooling the reaction mixture to from 40 to 55° C., optionally over the course of at least 30 minutes;
d) further cooling the reaction mixture to from 0 to 30° C. and stirring the mixture for a predefined amount of time thereby obtaining a solid;
e) adding further of the alcoholic solvent to the reaction mixture; and
f) collecting the solid by filtration and optionally drying the solid in vacuo.

In some embodiments, the alcoholic solvent is ethanol.

In some embodiments, the reaction mixture in b) is heated to from 70 to 80° C., such as 78° C.

In some embodiments, the predefined amount of time in b) is at least 15 minutes. In some embodiments, the predefined amount of time in b) is from 15 minutes to 2 hours, for example 30 minutes.

In some embodiments, the predefined amount of time in d) is from 30 minutes to 24 hours. In some embodiments, the predefined amount of time in d) is at least 1 hour. In some embodiments, the predefined amount of time in d) is from 1 hour to 5 hours.

In some embodiments, the reaction mixture in d) is cooled to from 5 to 25° C., such as 5° C., such as 6° C., such as 7° C., such as 8° C., such as 9° C., such as 10° C., such as 11° C., such as 12° C., such as 13° C., such as 14° C., such as 15° C., such as 16° C., such as 17° C., such as 18° C., such as 19° C., such as 20° C., such as 21° C., such as 22° C., such as 23° C., such as 24° C., or such as 25° C.

In some embodiments, from 4 to 10 volumes of the alcoholic solvent is used in the method for isolating the compound of formula Pg-O-IIIA-Pg* from a reaction mixture.

### Methods for Preparing the Final Compounds

The methods disclosed herein are particularly useful for preparing Pg-O-IIIA-Pg*, a key intermediate in the synthesis of buprenorphine, or a salt thereof. Hence, the present disclosure provides a method for preparing buprenorphine, or a salt thereof, comprising the method disclosed herein.

In some embodiments, the method disclosed herein comprises mixing Compound BnO-II-Bn with tert-butylmagnesium chloride and diglyme in a solvent system at a temperature within the range of from 5° C. to 60° C. and allowed to react for at from 15° C. to 60° C. at least 1 hour after mixing to provide Compound BnO-IIIA-Bn. In some embodiments, this method further comprises adding diglyme to the solvent system in a stochiometry with respect to the compound BnO-II-Bn of from 1 equivalent to 6 equivalents, such as from 2 equivalents to 6 equivalents, such as 4 equivalents.

In some embodiments, the solvent system comprises a) an ethereal solvent, such as diethyl ether or cyclopentyl methyl ether, and b) a nonpolar solvent, such as toluene.

In some embodiments, the method is provided wherein a) the tert-butylmagnesium chloride and diglyme is mixed in an ethereal solvent and optionally a non-polar solvent; and Compound BnO-II-Bn is added to a nonpolar solvent, such as toluene, and optionally also an ethereal solvent and then b) the compound BnO-II-Bn in the nonpolar solvent is added to the ethereal solvent of a).

In some embodiments, the method for preparing buprenorphine or a salt thereof comprises use of a solvent system comprising cyclopentyl methyl ether (CPME). In some embodiments, the solvent system comprises cyclopentyl methyl ether (CPME) and toluene.

In some embodiments, the method for preparing buprenorphine or a salt thereof further comprises the method for isolating a compound of formula Pg-O-IIIA-Pg* as defined herein.

Synthesis of Buprenorphine from Nororipavine

The method of the present disclosure is in some embodiments employed as a start in the synthesis of buprenorphine or analogues thereof starting from nororipavine, also referred to herein as compound HO-I-H. For this purpose, the present disclosure incorporates the synthesis steps and conditions disclosed in WO 2021/144362.

The disclosure further relates to methods for preparing buprenorphine:

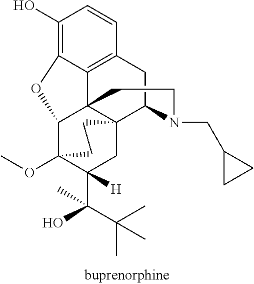 [SMILES: CO[C@]12CC[C@@]3(C[C@@H]1[C@](C)(O)C(C)(C)C)[C@H]1Cc4ccc(O)c5c4[C@@]3(CCN1CC1CC1)[C@H]2O5]

In various aspects and embodiments, the methods comprise a series of reaction steps to prepare buprenorphine from a compound of Formula I:

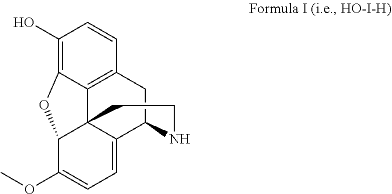 [SMILES: COC1=CC=C2[C@H]3Cc4ccc(O)c5c4[C@@]2(CCN3)[C@H]1O5]

Such methods provide an improved route to buprenorphine that can be shorter, more efficient, and/or produce less toxic waste than, e.g., current commercial routes to buprenorphine. As a result, these aspects and embodiments can be well-suited for commercial (e.g., kg-scale) production of buprenorphine. Further, such methods advantageously avoid the harsh conditions and/or toxic byproducts of an N-demethylation step and can accordingly be particularly well-suited for producing buprenorphine on a commercial, e.g., kg, scale.

In various aspects and embodiments, the methods comprise a series of reaction steps to prepare buprenorphine from a compound of Formula I-H:

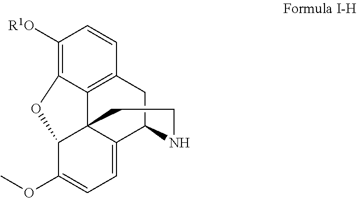

wherein R¹ is H (Compound HO-I-H; nororipavine).

In some embodiments, the methods comprise in a first solvent system S1 comprising a polar protic solvent reacting a compound of Formula I-H with benzyl halide, benzyl sulfonate, or activated benzyl alcohol to provide a compound of Formula I-Bn:

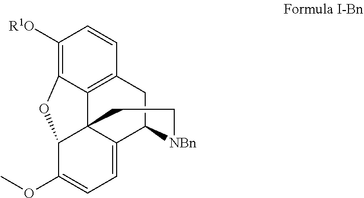

wherein R¹ is benzyl (Compound BnO-I-Bn). A preparation of Compound BnO-I-Bn, as an intermediate towards noroxymorphone and ultimately towards naltrexone and naloxone, was described in Helv. Chim. Acta 92:1359-65 (2009).

In some embodiments, the methods comprise in a second solvent system S2 comprising a polar protic solvent reacting a compound of Formula I-Bn with methyl vinyl ketone to provide a compound of Formula II-Bn:

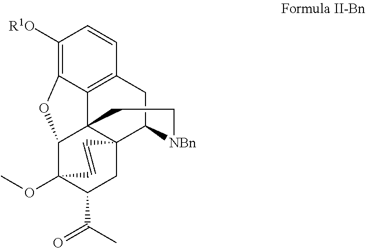

wherein R¹ is benzyl (Compound BnO-II-Bn).

In some embodiments, the methods comprise in a third solvent system S3 comprising a nonpolar solvent reacting a compound of Formula II-Bn with tert-butylmagnesium halide to provide a compound of Formula IIIA-Bn:

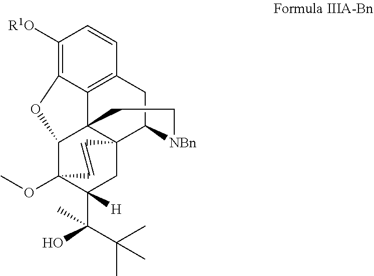

wherein R¹ is benzyl (Compound BnO-IIIA-Bn).

In some embodiments, the methods comprise reacting a compound of Formula IIIA-Bn with H₂ in the presence of a hydrogenation catalyst to provide a compound of Formula IV-H:

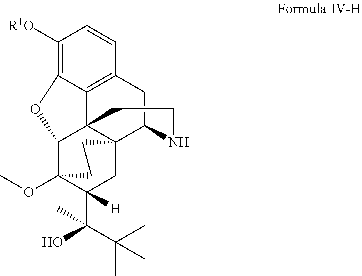

wherein R¹ is H (Compound HO-IV-H; norbuprenorphine).

In some embodiments, the methods comprise reacting a compound of Formula IV-H (e.g., Compound HO-IV-H) with cyclopropane carboxaldehyde followed by a hydride source; or reacting a compound of Formula IV-H (e.g., Compound HO-IV-H) with cyclopropanecarboxylic acid halide followed by a reducing agent; or reacting a compound of Formula IV-H (e.g., Compound HO-IV-H) with cyclopropylmethyl halide or activated cyclopropane methanol; to provide buprenorphine.

### Formula I-H→Formula I-Bn

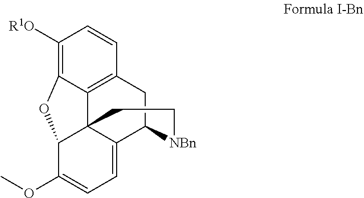

| R¹ of Formula I-Bn | Compound |
|---|---|
| Bn | Compound BnO-I-Bn |

Step (i)(F)

In some embodiments, reacting a compound of Formula I-H with benzyl halide, benzyl sulfonate, or activated benzyl alcohol (e.g., activated with a sulfonate group such as a p-toluene sulfonyl group or a methyl sulfonyl group, or with triphenylphosphine) in a first solvent system S1 comprising a polar protic solvent provides a compound of Formula I-Bn. In certain embodiments, reacting Compound HO-I-H with benzyl halide, benzyl sulfonate, or activated benzyl alcohol provides Compound BnO-I-Bn.

In some embodiments, the benzyl halide is benzyl chloride or benzyl bromide. In some embodiments, the reaction is performed in the presence of a strong base, e.g., an alkali metal hydroxide. In some embodiments, the first solvent system S1 has a dielectric constant of at least about 12, or at least about 13, or at least about 14. In various other embodiments as described herein, the dielectric constant of S1 is at least about 15, or at least about 16, or at least about 18, or at least about 20.

In certain embodiments as otherwise described herein, S1 comprises at least about 50 vol. % of at least one protic solvent having a dielectric constant of at least about 12. In various other embodiments, the at least one protic solvent is present in an amount of at least 60 vol. %, or at least 70 vol. %, or at least 75 vol. %, or at least 80 vol. %, or at least 90 vol. %, or at least 95 vol. %. In certain embodiments, the at least one protic solvent has a dielectric constant of at least 13, or at least 14, or at least 15, or at least 16, or at least 18, or at least 20.

In some embodiments, S1 comprises at least one protic solvent having a polarity index of at least about 3, or at least about 3.5, or at least about 3.75, or at least about 4. As used herein, the solvent polarity index of a solvent can be determined according to Snyder, e.g., as reported in Snyder, L. R. “Classification of the Solvent Properties of Common Liquids.” J. Chromatogr. (1978) 16:6, 223-234. In various embodiments, S1 comprises at least about 50 vol. %, or at least about 75 vol. %, or at least about 90 vol. % of at least one protic solvent having a polarity index of at least about 3, or at least 3.5, or at least 3.75, or at least 4.

In some embodiments as otherwise described herein, S1 comprises a C₁-C₄ alcohol (e.g., methanol, ethanol, n-propanol, isopropanol, n-butanol, or sec-butanol) and optionally water. In various embodiments, S1 comprises about 50-100 vol. % isopropanol and 0-50 vol. % water. In some embodiments, the benzyl halide, benzyl sulfonate, or activated benzyl alcohol is reacted at a temperature within the range of about −20° C. to about 40° C., e.g., about −20° C. to about 35° C., or about −20° C. to about 30° C., or about −20° C. to about 25° C., or about −20° C. to about 20° C., or about −20° C. to about 15° C., or about −20° C. to about 10° C., or about −20° C. to about 5° C., or about −20° C. to about 0° C., or about −15° C. to about 40° C., or about −10° C. to about 40° C., or about −5° C. to about 40° C., or about 0° C. to about 40° C., or about 5° C. to about 20° C., or about 10° C. to about 40° C., or about 15° C. to about 40° C., or about 20° C. to about 40° C., or about −15° C. to about 35° C., or about −10° C. to about 30° C., or about −5° C. to about 25° C., or about 0° C. to about 20° C., or about 5° C. to about 15° C. In some embodiments, the benzyl halide, benzyl sulfonate, or activated benzyl alcohol is reacted for a period of time within the range of about 1 h to about 2 days, e.g., 2 h to about 2 days, 3 h to about 2 days, 6 hours to about 2 days, about 12 hours to about 2 days, or about 18 hours to about 2 days, or about 1 day to about 2 days, or about 1.25 days to about 2 days, or about 1.5 days to about 2 days, or about 6 hours to about 1.75 days, or about 6 hours to about 1.5 days, or about 6 hours to about 1.25 days, or about 6 hours to about 1 day, or about 6 hours to about 18 hours, or about 12 hours to about 1.75 days, or about 18 hours to about 1.5 days, or about 1 h to about 1 day, or about 1 h to about 12 h, or about 1 h to about 6 h, or about 1 h to about 4 h.

### Formula I-Bn→Formula II-Bn

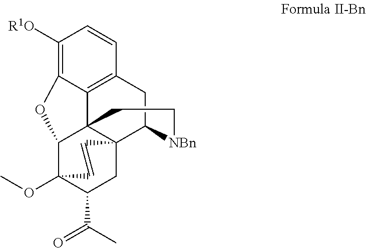

| R¹ of Formula II-Bn | Compound |
|---|---|
| Bn | Compound BnO-II-Bn |

Step (ii)(B)

In some embodiments, reacting a compound of Formula I-Bn with methyl vinyl ketone in a second solvent system S2 comprising a polar protic solvent provides a compound of Formula II-Bn. In certain embodiments, reacting Compound BnO-I-Bn with methyl vinyl ketone provides Compound BnO-II-Bn.

In some embodiments, the methyl vinyl ketone is reacted at a temperature within the range of about 40° C. to about 120° C., e.g., about 45° C. to about 120° C., or about 50° C. to about 120° C., or about 55° C. to about 120° C., or about 60° C. to about 120° C., or about 65° C. to about 120° C., or about 70° C. to about 120° C., or about 75° C. to about 120° C., or about 80° C. to about 120° C., or about 85° C. to 120° C., or about 90° C. to about 120° C., or about 40° C. to about 115° C., or about 40° C. to about 110° C., or about 40° C. to about 105° C., or about 40° C. to about 100° C., or about 40° C. to about 95° C., or about 40° C. to about 90° C., or about 40° C. to about 85° C., or about 40° C. to about 80° C., or about 40° C. to about 75° C., or about 40° C. to about 70° C., or about 45° C. to about 115° C., or about 50° C. to about 110° C., or about 55° C. to about 105° C., or about 60° C. to about 100° C., or about 65° C. to about 95° C., or about 70° C. to about 90° C.

In some embodiments, the methyl vinyl ketone is reacted for a period of time within the range of about 2 hours to about 2 days, e.g., about 4 hours to about 2 days, or about 6 hours to about 2 days, or about 12 hours to about 2 days, or about 18 hours to about 2 days, or about 1 days to about 2 days, or about 1.25 days to about 2 days, or about 1.5 days to about 2 days, or about 2 hours to about 1.75 days, or about 2 hours to about 1.5 days, or about 2 hours to about 1.25 days, or about 2 hours to about 1 day, or about 2 hours to about 18 hours, or about 2 hours to about 12 hours, or about 4 hours to about 1.75 days, or about 6 hours to about 1.5 days, or about 12 hours to about 1.25 days, or about 18 hours to about 1 day.

In certain embodiments as otherwise described herein, the reaction of (ii)(B) is carried out under oxygen, e.g., a mixture of inert gas and oxygen having a different composition than that of air. In certain embodiments, the reaction is carried out in an atmosphere wherein inert gas (e.g., argon) is present as greater than 5 vol. % (e.g., greater than 20 vol. %, or greater than 50 vol. %), and and oxygen is present as less than 25 vol. % (e.g., less than 21 vol. %, or less than 20 vol. %, or less than 10 vol. %, or less than 5 vol. %). In certain embodiments, the reaction is carried out in an atmosphere wherein oxygen is present at between 1 vol. % and about 21 vol. %, or between 3 vol. % and 20 vol. %, or between 5 vol. % and 20 vol. %, or between 10 vol. % and 20 vol. %, or between 1 vol. % and 20 vol. %, or between 1 vol. % and 15 vol. %, or between 1 vol. % and 10 vol. %, or between 1 vol. % and 7 vol. %, or between 1 vol. % and 5 vol. %. In certain other embodiments, the reaction is performed in substantially inert atmosphere (e.g., oxygen is present at less than 0.1 vol. %, or less than 0.01 vol. %, or less than 0.001 vol. %).

In certain other embodiments, the reaction of (ii)(B) is carried out under a mixture of gases approximately the same as air (e.g., dry air). In other embodiments, the reaction is carried out wherein the ratio of inert gas to gaseous oxygen is approximately 79 vol. % to 21 vol. %.

It has been found that the use of some amount of oxygen in the atmosphere of the reaction (ii)(B) serves to increase the yield of the reaction. Without wishing to be bound by theory, it is presently believed that trace oxygen prevents methyl vinyl ketone polymerization, allowing for additional methyl vinyl ketone monomers to be present as reactants. This reduces the equivalents of methyl vinyl ketone necessary to propel the reaction to completion, and can also increase the reaction yield. Beyond reducing the need for excess methyl vinyl ketone, providing a reaction atmosphere containing at least some oxygen generally requires less rigorous reaction condition and equipment, especially at scale, as rigorous oxygen exclusion is no longer required. Together, this development allows for a more efficient synthetic protocol and enhanced reaction yields with lower precursor and capital expenditures.

In some embodiments, the second solvent system S2 has a dielectric constant of at least about 12, or at least about 13, or at least about 14. In various other embodiments as described herein, the dielectric constant of S2 is at least about 15, or at least about 16, or at least about 18, or at least about 20.

In certain embodiments as otherwise described herein, S2 comprises at least about 50 vol. % of at least one protic solvent having a dielectric constant of at least about 12. In various other embodiments, the at least one protic solvent is present in an amount of at least 60 vol. %, or at least 70 vol. %, or at least 75 vol. %, or at least 80 vol. %, or at least 90 vol. %, or at least 95 vol. %. In certain embodiments, the at least one protic solvent has a dielectric constant of at least 13, or at least 14, or at least 15, or at least 16, or at least 18, or at least 20.

In some embodiments, S2 comprises at least one protic solvent having a polarity index of at least about 3, or at least about 3.5, or at least about 3.75, or at least about 4. In various embodiments, S2 comprises at least about 50 vol. %, or at least about 75 vol. %, or at least about 90 vol. % of at least one protic solvent having a polarity index of at least about 3, or at least 3.5, or at least 3.75, or at least 4.

In some embodiments as otherwise described herein, S2 comprises a C₁-C₄ alcohol (e.g., methanol, ethanol, n-propanol, isopropanol, n-butanol, or sec-butanol) and optionally water. In various embodiments, S2 comprises about 50-100 vol. % isopropanol and 0-50 vol. % water. In certain desirable embodiments, S2 has substantially the same composition as S1, described above.

### Telescoped Synthesis: Formula I-H→Formula II-Bn

In certain desirable embodiments, reactions (i)(F) and (ii)(B) can be performed sequentially, advantageously without intervening purification and without substantial removal of solvent system S1. In certain embodiments as otherwise described herein, S2 has substantially the same composition as S1. In such embodiments, the reactants and purification steps of step (ii)(B) comprise the crude reaction product of step (i)(F). Accordingly, in certain embodiments as otherwise described herein, the methyl vinyl ketone of step (ii)(B) is added to a crude reaction product of step (i)(F), the crude reaction product comprising solvent S1 and Compound BnO-II-Bn.

As noted above, the reaction steps of (ii)(B) can be carried out without substantial removal of solvent or purification (e.g. chromatography). However, the person of ordinary skill in the art will appreciate that it may be necessary to adjust the pH of the crude reaction product of step (i)(F) before performing step (ii)(B). The pH may be adjusted with a wide variety of acids known in the art.

For example, in certain embodiments, the pH is adjusted with the addition of acetic acid or hydrochloric acid. For example, the pH may be adjusted with a water-diluted acid such as 10% acetic acid, or 10% hydrochloric acid. In various embodiments as otherwise described herein, the pH is adjusted to be about neutral, e.g., between 6 and 8.

### Formula II-Bn→Formula IIIA-Bn

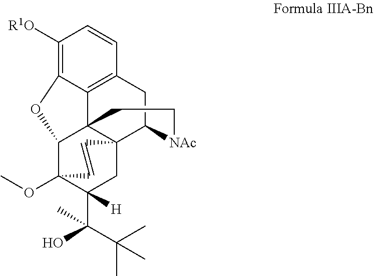

| R¹ of Formula IIIA-Bn | Compound |
|---|---|
| Bn | Compound BnO-IIIA-Bn |

Step (iii)(D)

The present disclosure provides improved conditions for step (iii)(D) as disclosed herein. In some embodiments, the conditions optionally also include reacting a compound of Formula II-Bn with a tert-butylmagnesium compound in a third solvent system S3 comprising a nonpolar solvent providing a compound of Formula IIIA-Bn. In certain embodiments, reacting Compound BnO-II-Bn with a tert-butylmagnesium compound provides Compound BnO-IIIA-Bn.

In some embodiments, the tert-butylmagnesium compound is a tert-butylmagnesium halide. For example, the tert-butylmagnesium compound is tert-butylmagnesium chloride or tert-butylmagnesium bromide. In some embodiments, the reaction is performed in a solvent comprising a nonpolar solvent, e.g., tert-butylmethyl ether, 2-methyl-tetrahydrofuran, diethyl ether, dimethoxymethane, benzene, toluene, or a mixture of thereof.

In some embodiments, the tert-butylmagnesium compound is reacted at a temperature within the range of about 15° C. to about 100° C., e.g., about 20° C. to about 100° C., or about 25° C. to about 100° C., or about 30° C. to about 100° C., or about 15° C. to about 95° C., or about 15° C. to about 90° C., or about 15° C. to about 85° C., or about 20° C. to about 95° C., or about 25° C. to about 90° C. In some embodiments, the tert-butylmagnesium halide is reacted for a period of time within the range of about 30 minutes to about 8 hours, e.g., about 1 hours to about 8 hours, or about 1.5 hours to about 8 hours, or about 2 hours to about 8 hours, or about 2.5 hours to about 8 hours, or about 3 hours to about 8 hours, or about 3.5 hours to about 8 hours, or about 4 hours to about 8 hours, or about 4.5 hours to about 8 hours, or about 5 hours to about 8 hours, or about 30 minutes to about 7.5 hours, or about 30 minutes to about 7 hours, or about 30 minutes to about 6.5 hours, or about 30 minutes to about 6 hours, or about 30 minutes to about 5.5 hours, or about 30 minutes to about 5 hours, or about 30 minutes to about 4.5 hours, or about 30 minutes to about 4 hours, or about 30 minutes to about 3.5 hours, or about 1 hour to about 7.5 hours, or about 1.5 hours to about 7 hours, or about 2 hours to about 6.5 hours, or about 2.5 hours to about 6 hours, or about 3 hours to about 5.5 hours.

As described above, the third solvent system S3 comprises a nonpolar solvent. In certain embodiments as otherwise described herein, the third solvent system S3 comprises at least one at least one nonpolar solvent having a dielectric constant of at most about 8, or at most about 7, or at most about 6, or at most about 5, or at most about 4, or at most about 3. For example, in various embodiments, S3 comprises at least 60 vol. %, or at least 70 vol. %, or at least 75 vol. %, or at least 80 vol. %, or at least 90 vol. %, or at least 95 vol. % of the at least one nonpolar solvent having a dielectric constant of at most 8, or at most 7, or at most 6, or at most 5, or at most 4, or at most 3. In further embodiments, the nonpolar solvent that comprises S3 has a polarity index of less than 4, or less than 3, or less than 2, or less than 1. For example, in various embodiments, S3 comprises at least 60 vol. %, or at least 70 vol. %, or at least 75 vol. %, or at least 80 vol. %, or at least 90 vol. %, or at least 95 vol. % of the at least one nonpolar solvent has a polarity index of less than 4, or less than 3, or less than 2, or less than 1.

In certain desirable embodiments, polar solvents or solvents with large dielectric constants are not substantially present in S3, or are present in S3 in a relatively small amount. For example, in certain embodiments, S3 comprises less than about 20 vol. %, or less than about 10 vol. %, or less than about 5 vol. %, or less than about 1 vol. % of a total amount of solvents having a dielectric constant of greater than 4, or greater than 6, or greater than 8. In various embodiments as otherwise described herein, S3 comprises less than about 20 vol. %, or less than about 10 vol. %, or less than about 5 vol. %, or less than about 1 vol. % of a total amount of solvents having a polarity index of 2 or greater, or 3 or greater, or 4 or greater.

In some embodiments, S3 comprises 30-90 vol. % of one or more C₅-C₁₀ alkanes and/or C₅-C₁₀ cycloalkanes. In certain embodiments, the alkanes and/or cycloalkanes are substituted (e.g., perfluorocyclohexane, perfluorohexane, etc.). For example, in certain embodiments the one or more alkanes and/or cycloalkanes include cyclohexane. In other embodiments, the one or more alkanes and/or cycloalkanes is cyclohexane. For example, S3 may comprise 10-50 vol. % toluene (e.g., 20-50 vol. % toluene, or 30-50 vol. % toluene), 30-90 vol. % cyclohexane (e.g., 40-90 vol. % cyclohexane, or 40-70 vol. % cyclohexane), and up to 30 vol % tetrahydrofuran (e.g., up to 20 vol. % tetrahydrofuran, or up to 10 vol. % tetrahydrofuran, or up to 5 vol. % tetrahydrofuran).

It is known in the art that certain Grignard reagents (e.g., tert-butylmagnesium halide) disproportionate to the bis-alkyl and bis-halide species, with the disproportionation favored under certain reaction and solvent conditions (e.g. a more polar solvent). Without wishing to be bound by theory, the present inventors believe that the reaction conditions of step (iii)(D) described herein can increase the concentration of the bis adducts present in solution, advantageously improving the yield of Formula IIIA-Bn (e.g., Compound BnO-IIIA-Bn). Accordingly, in certain embodiments, the tert-butylmagnesium compound comprises one or both of a tert-butylmagnesium halide and di-tert-butylmagnesium. For example, in some embodiments, the tert-butylmagnesium compound comprises a tert-butylmagnesium halide and di-tert-butylmagnesium. In certain embodiments, a proportion of the magnesium dihalide (e.g., magnesium dichloride) precipitates from solution. For example, substantially all of the magnesium dihalide may precipitate from solution. Alternatively, in certain other embodiments, there is essentially no precipitate formed from the Grignard reagent.

### Formula IIIA-Bn→Formula IV-H

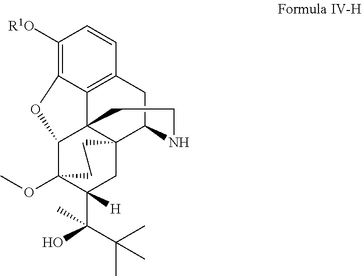

| R¹ of Formula IV-H | Compound |
|---|---|
| H | HO-IV-H |

Step (iv)(C)

In some embodiments, reacting a compound of Formula IIIA-Bn with H₂ in the presence of a hydrogenation catalyst provides a compound of Formula IV-Bn. In certain embodiments, reacting Compound BnO-IIIA-Bn with H₂ in the presence of a hydrogenation catalyst provides Compound HO-IV-H.

In some embodiments, the hydrogenation catalyst comprises nickel, palladium, platinum, rhodium, or ruthenium. In some embodiments, the hydrogenation catalyst comprises platinum or palladium, supported on carbon. In some embodiments, the reaction is performed in a solvent comprising a polar protic or aprotic solvent, e.g., n-butanol, isopropanol, ethanol, methanol, N-methylpyrrolidone, tetrahydrofuran, ethyl acetate, acetone, dimethylformamide, acetonitrile, dimethylsulfoxide, propylene carbonate, or a mixture thereof.

In some embodiments, the hydrogen is reacted at a temperature within the range of about 15° C. to about 120° C., e.g., about 20° C. to about 120° C., or about 30° C. to about 120° C., or about 40° C. to about 120° C., or about 15° C. to about 115° C., or about 20° C. to about 110° C., or about 30° C. to about 105° C., or about 40° C. to about 115° C., or about 50° C. to about 110° C. In some embodiments, the hydrogen is reacted for a period of time within the range of about 6 hours to about 3 days, e.g., about 12 hours to about 3 days, or about 18 hours to about 3 days, or about 1 day to about 3 days, or about 1.25 days to about 3 days, or about 1.5 days to about 3 days, or about 6 hours to about 2.75 days, or about 6 hours to about 2.5 days, or about 6 hours to about 2.25 days, or about 6 hours to about 2 day, or about 6 hours to about 36 hours, or about 12 hours to about 2.5 days, or about 24 hours to about 2 days. In some embodiments, the hydrogen is reacted at a pressure within the range of about 1 atm to about 3 atm, e.g., about 1.25 atm to about 3 atm, or about 1.5 atm to about 3 atm, or about 1.75 atm to about 3 atm, or about 2 atm to about 3 atm, or about 1 atm to about 2.75 atm, or about 1 atm to about 2.5 atm, or about 1 atm to about 2.25 atm, or about 1 atm to about 2 atm, or about 1.25 atm to about 2.75 atm, or about 1.5 atm to about 2.5 atm, or about 1.75 atm to about 2.25 atm.

### Formula IV-H→Formula IV-MCP

Step (v)(A1)

In some embodiments, reacting a compound of Formula IV-H with cyclopropane carboxaldehyde followed by a hydride source provides a compound of Formula IV-MCP. In certain embodiments, reacting Compound HO-IV-H with cyclopropane carboxaldehyde followed by a hydride source provides buprenorphine.

In some embodiments, the hydride source is formic acid, hydrogen, sodium cyanoborohydride, sodium borohydride, or sodium triacetoxy borohydride. In some embodiments, the hydride source is formic acid. In some embodiments, the reaction is catalyzed by a ruthenium(I) complex or a ruthenium(II) complex, e.g., a dichloro(p-cymene)ruthenium(II) dimer. In some embodiments, the reaction is performed in a solvent comprising a polar aprotic solvent, e.g., N-methylpyrrolidone, tetrahydrofuran, ethyl acetate, acetone, dimethylformamide, acetonitrile, dimethylsulfoxide, propylene carbonate, or a mixture thereof. In some embodiments, the reaction is performed in the presence of a trialkylamine, e.g., triethylamine, diisopropylethylamine, 4-methyl-morpholine, or N-methyl-piperidine.

In some embodiments, the cyclopropane carboxaldehyde is reacted at a temperature within the range of about 30° C. to about 90° C., e.g., about 35° C. to about 90° C., or about 40° C. to about 90° C., or about 45° C. to about 90° C., or about 50° C. to about 90° C., or about 55° C. to about 90° C., or about 60° C. to about 90° C., or about 65° C. to about 90° C., or about 70° C. to about 90° C., or about 30° C. to about 85° C., or about 30° C. to about 80° C., or about 30° C. to about 75° C., or about 30° C. to about 70° C., or about 30° C. to about 65° C., or about 30° C. to about 60° C., or about 30° C. to about 55° C., or about 30° C. to about 50° C., or about 35° C. to about 85° C., or about 40° C. to about 80° C., or about 45° C. to about 75° C., or about 50° C. to about 70° C., or about 55° C. to about 65° C. In some embodiments, the cyclopropane carboxaldehyde is reacted for a period of time within the range of about 30 minutes to about 5 hours, e.g., about 1 hour to about 5 hours, or about 1.5 hours to about 5 hours, or about 2 hours to about 5 hours, or about 2.5 hours to about 5 hours, or about 3 hours to about 5 hours, or about 3.5 hours to about 5 hours, or about 4 hours to about 5 hours, or about 30 minutes to about 4.5 hours, or about 30 minutes to about 4 hours, or about 30 minutes to about 3.5 hours, or about 30 minutes to about 3 hours, or about 30 minutes to about 2.5 hours, or about 30 minutes to about 2 hours, or about 30 minutes to about 1.5 hours.

Step (v)(A2)

In some embodiments, reacting a compound of Formula IV-H with cyclopropanecarboxylic acid halide followed by a reducing agent provides a compound of Formula IV-MCP. In certain embodiments, reacting Compound HO-IV-H with cyclopropanecarboxylic acid halide followed by a reducing agent provides buprenorphine.

In some embodiments, the cyclopropanecarboxylic acid halide is cyclopropanecarboxylic acid chloride, cyclopropanecarboxylic acid anhydride, cyclopropanecarboxylic acid bromide, or an activated cyclopropanecarboxylic acid (e.g., an activated cyclopropanecarboxylic acid formed by reaction with an alcohol such as pentafluorophenol, 4-nitrophenol, N-hydroxysuccinimide, N-hydroxymaleimide, 1-Hydroxybenzotriazole, or 1-hydroxy-7-azabenzotriazole). In some embodiments, the reducing agent is LiAlH₄ or NaBH₄. In some embodiments, the reaction with cyclopropanecarboxylic acid halide is performed in a solvent comprising a nonpolar solvent, e.g., dichloromethane, chloroform, toluene, 1,4-dioxane, diethyl ether, benzene, or a mixture thereof. In some embodiments, the reaction with a reducing agent is performed in a solvent comprising a polar acetate, acetone, aprotic solvent, e.g., N-methylpyrrolidone, tetrahydrofuran, ethyl dimethylformamide, acetonitrile, dimethylsulfoxide, propylene carbonate, or a mixture thereof.

In some embodiments, the cyclopropanecarboxylic acid halide is reacted at a temperature within the range of about −20° C. to about 40° C., e.g., about −20° C. to about 35° C., or about −20° C. to about 30° C., or about −20° C. to about 25° C., or about −20° C. to about 20° C., or about −20° C. to about 15° C., or about −20° C. to about 10° C., or about −20° C. to about 5° C., or about −20° C. to about 0° C., or about −15° C. to about 40° C., or about −10° C. to about 40° C., or about −5° C. to about 40° C., or about 0° C. to about 40° C., or about 5° C. to about 20° C., or about 10° C. to about 40° C., or about 15° C. to about 40° C., or about 20° C. to about 40° C., or about −15° C. to about 35° C., or about −10° C. to about 30° C., or about −5° C. to about 25° C., or about 0° C. to about 20° C., or about 5° C. to about 15° C. In some embodiments, the cyclopropanecarboxylic acid halide is reacted for a period of time within the range of about 6 hours to about 2 days, e.g., about 12 hours to about 2 days, or about 18 hours to about 2 days, or about 1 day to about 2 days, or about 1.25 days to about 2 days, or about 1.5 days to about 2 days, or about 6 hours to about 1.75 days, or about 6 hours to about 1.5 days, or about 6 hours to about 1.25 days, or about 6 hours to about 1 day, or about 6 hours to about 18 hours, or about 12 hours to about 1.75 days, or about 18 hours to about 1.5 days. In some embodiments, the reducing agent is reacted at a temperature within the range of about 35° C. to about 85° C., e.g., about 40° C. to about 85° C., or about 45° C. to about 85° C., or about 50° C. to about 85° C., or about 55° C. to about 85° C., or about 60° C. to about 85° C., or about 65° C. to about 85° C., or about 35° C. to about 80° C., or about 35° C. to about 75° C., or about 35° C. to about 70° C., or about 35° C. to about 65° C., or about 35° C. to about 60° C., or about 35° C. to about 55° C., or about 40° C. to about 80° C., or about 45° C. to about 75° C., or about 50° C. to about 70° C., or about 55° C. to about 65° C. In some embodiments, the reducing agent is reacted for a period of time within the range of about 5 minutes to about 3 hours, e.g., or about 10 minutes to about 3 hours, or about 15 minutes to about 3 hours, or about 30 minutes to about 3 hours, or about 45 minutes to about 3 hours, or about 1 hour to about 3 hours, or about 1.25 hours to about 3 hours, or about 1.5 hours to about 3 hours, or about 1.75 hours to about 3 hours, or about 2 hours to about 3 hours, or about 5 minutes to about 2.75 hours, or about 5 minutes to about 2.5 hours, or about 5 minutes to about 2.25 hours, or about 5 minutes to about 2 hours, or about 5 minutes to about 1.75 hours, or about 5 minutes to about 1.5 hours, or about 5 minutes to about 1.25 hours, or about 5 minutes to about 1 hour, or about 10 minutes to about 2.75 hours, or about 15 minutes to about 2.5 hours, or about 30 minutes to about 2.25 hours, or about 45 minutes to about 2 hours, or about 1 hour to about 1.75 hours.

Step (v)(A3)

In some embodiments, reacting a compound of Formula IV-H with cyclopropylmethyl halide or activated cyclopropane methanol (e.g., activated with a sulfonate group such as a p-toluene sulfonyl group or a methyl sulfonyl group, or with triphenylphosphine) provides a compound of Formula IV-MCP. In certain embodiments, reacting Compound HO-IV-H with cyclopropylmethyl halide or activated cyclopropane methanol provides buprenorphine.

In some embodiments, the cyclopropylmethyl halide is cyclopropylmethyl chloride or

cyclopropylmethyl bromide. In some embodiments, the reaction is performed in the presence of a trialkylamine, e.g., triethylamine, diisopropylethylamine, 4-methyl-morpholine, or N-methyl-piperidine. In some embodiments, the reaction is performed in a solvent comprising a polar protic solvent, e.g., n-butanol, isopropanol, ethanol, methanol, water, or a mixture thereof.

In some embodiments, the cyclopropylmethyl halide or activated cyclopropane methanol is reacted at a temperature within the range of about 40° C. to about 120° C., e.g., about 45° C. to about 120° C., or about 50° C. to about 120° C., or about 55° C. to about 120° C., or about 60° C. to about 120° C., or about 65° C. to about 120° C., or about 70° C. to about 120° C., or about 75° C. to about 120° C., or about 80° C. to about 120° C., or about 85° C. to 120° C., or about 90° C. to about 120° C., or about 40° C. to about 115° C., or about 40° C. to about 110° C., or about 40° C. to about 105° C., or about 40° C. to about 100° C., or about 40° C. to about 95° C., or about 40° C. to about 90° C., or about 40° C. to about 85° C., or about 40° C. to about 80° C., or about 40° C. to about 75° C., or about 40° C. to about 70° C., or about 45° C. to about 115° C., or about 50° C. to about 110° C., or about 55° C. to about 105° C., or about 60° C. to about 100° C., or about 65° C. to about 95° C., or about 70° C. to about 90° C. In some embodiments, the cyclopropylmethyl halide or activated cyclopropane methanol is reacted for a period of time within the range of about 30 minutes to about 6 hours, e.g., about 1 hours to about 6 hours, or about 1.5 hours to about 6 hours, or about 2 hours to about 6 hours, or about 2.5 hours to about 6 hours, or about 3 hours to about 6 hours, or about 3.5 hours to about 6 hours, or about 4 hours to about 6 hours, or about 30 minutes to about 5.5 hours, or about 30 minutes to about 5 hours, or about 30 minutes to about 4.5 hours, or about 30 minutes to about 4 hours, or about 30 minutes to about 3.5 hours, or about 30 minutes to about 3 hours, or about 30 minutes to about 2.5 hours, or about 1 hours to about 5.5 hours, or about 1.5 hours to about 5 hours, or about 2 hours to about 4.5 hours, or about 2.5 hours to about 4 hours.

### Formula I-H→Buprenorphine

In another aspect, the method of preparing buprenorphine comprises the series of steps provided in Table A:

TABLE A
5-step buprenorphine route

| No. | Substrate | Step | Product |
|---|---|---|---|
| I | Compound HO-I-H | (F) | Compound BnO-I-Bn |
| ii | Compound BnO-I-Bn | (B) | Compound BnO-II-Bn |
| iii | Compound BnO-II-Bn | (D) | Compound BnO-IIIA-Bn |
| iv | Compound BnO-IIIA-Bn | (C) | Compound HO-IV-H |
| v | Compound HO-IV-H | (A1), (A2), or (A3) | buprenorphine |

The person of ordinary skill in the art will appreciate that additional steps such as, for example, purification (e.g., crystallization) or formation of an addition salt (e.g., formation of buprenorphine-HCl) may be included in the methods of the disclosure as otherwise described herein.

In some embodiments, the method disclosed herein is provided further comprising one, more or all of the steps of:
step (i)(F): in a solvent system S1 comprising a polar protic solvent, reacting Compound HO-I-H

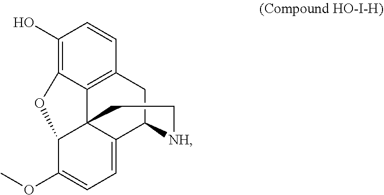 [SMILES: COC1=CC=C2[C@H]3Cc4ccc(O)c5c4[C@@]2(CCN3)[C@H]1O5]

with benzyl halide, benzyl sulfonate, or activated benzyl alcohol to provide Compound BnO-I-Bn:

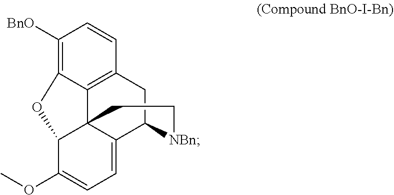 [SMILES: COC1=CC=C2[C@H]3Cc4ccc(OCc5ccccc5)c5c4[C@@]2(CCN3Cc2ccccc2)[C@H]1O5]

step (ii)(B): in a solvent system S2 comprising a polar protic solvent, reacting Compound BnO-I-Bn with methyl vinyl ketone to provide Compound BnO-II-Bn:

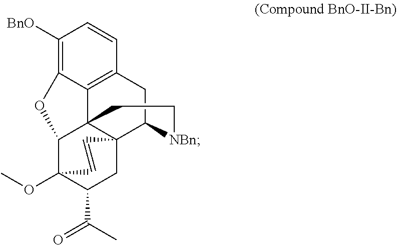 [SMILES: CO[C@]12C=C[C@@]3(C[C@@H]1C(C)=O)[C@H]1Cc4ccc(OCc5ccccc5)c5c4[C@@]3(CCN1Cc1ccccc1)[C@H]2O5]

step (iii)(D): in a solvent system S3 comprising a nonpolar solvent, reacting Compound BnO-II-Bn with a tert-butylmagnesium compound to provide Compound BnO-IIIA-Bn:

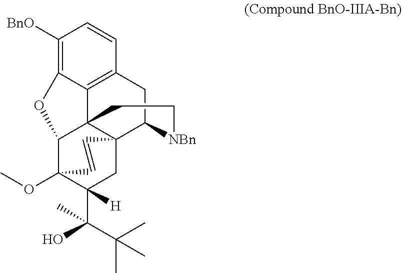 [SMILES: CO[C@]12C=C[C@@]3(C[C@@H]1[C@](C)(O)C(C)(C)C)[C@H]1Cc4ccc(OCc5ccccc5)c5c4[C@@]3(CCN1Cc1ccccc1)[C@H]2O5]

Step (iv)(C): reacting Compound BnO-IIIA-Bn with H₂ in the presence of a hydrogenation catalyst to provide a compound of Compound HO-IV-H:

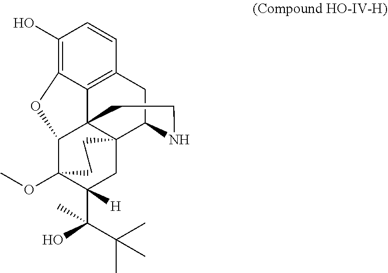 [SMILES: CO[C@]12CC[C@@]3(C[C@@H]1[C@](C)(O)C(C)(C)C)[C@H]1Cc4ccc(O)c5c4[C@@]3(CCN1)[C@H]2O5]

Step (v)(A1): reacting Compound HO-IV-H with cyclopropane carboxaldehyde followed by a hydride source; or
Step (v)(A2): reacting Compound HO-IV-H with cyclopropanecarboxylic acid halide followed by a reducing agent; or
Step (v)(A3): reacting Compound HO-IV-H with cyclopropylmethyl halide or activated cyclopropane methanol;
to provide buprenorphine.

### EXAMPLES

### Materials and Methods

Chemicals used in the examples herein, e.g. for buffers and solvents, are commercial products of at least reagent grade.

### Example 1—Optimization of Reactions Conditions of the Grignard Addition

The present example studies the effect of varying the solvent, temperature, time, additive and further reaction conditions on the yield of the desired Grignard adduct, namely the(S)-stereoisomer of BnO-IIIA-Bn.

### Method

Unless otherwise stated, a solution of the ketone BnO-II-Bn in a solvent as specified in table 1-4 was added to the stirred Grignard solution over a time course and at a temperature specified under “Reaction Conditions” in tables 1-4.

The general reaction scheme is outlined below:

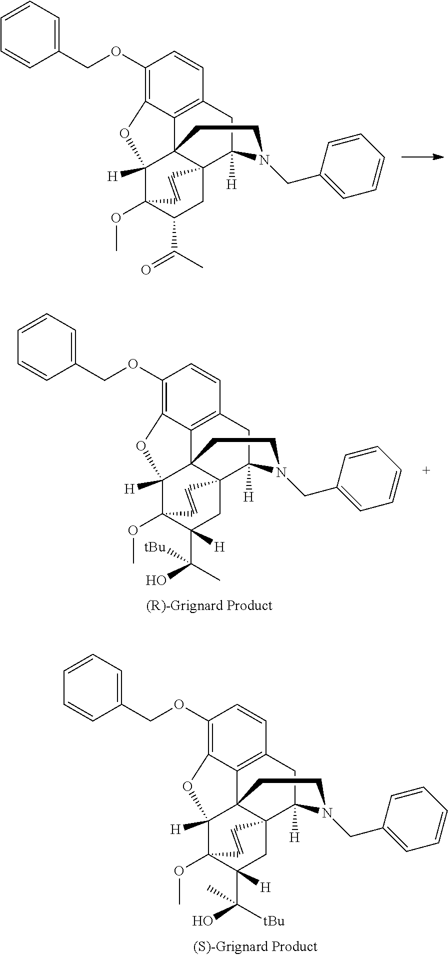

The side product referred to as “reduced” in the tables 1-7 below:

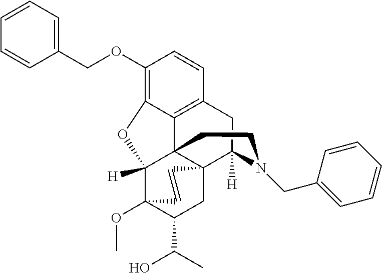

### Results

The below tables 1-7 demonstrate the conditions and results of Grignard reactions performed.

TABLE 1
Overview of outcome of Grignard reaction using different reaction conditions. All reactions were cooled
to −5° C. before quenching with NH4Cl then extraction work-up with EtOAc, washing with brine.

| | Grignard Solution | | | | | Crude Product Mixture^{a} | | | |
|---|---|---|---|---|---|---|---|---|---|
| | t-BuMgCl | LaCl₃•2LiCl | Diluent | | | | | % | |
| | (2N in | (0.6N in | (Cyclohexane | Ketone | | % addition^{b} | | SM/ | |
| entry | Et₂O) | THF) | or Toluene) | Solution | Reaction Conditions | (S) | (R) | enol | % reduced |
| AG- | 0.25 mL, | 0.122 mL | 5.0 mL | Toluene | Ketone → Grignard | 66 | — | 28 | 6 |
| 005 | (2.63 eq.) | (0.39 eq.) | Cyclohexane | (0.5 mL) | over 20 min, −25° C. | | | | |
| | | | | and | Warmed to −5° C., 1 h | | | | |
| | | | | Cyclohexane | | | | | |
| | | | | (0.5 mL) | | | | | |
| AG- | 0.50 mL, | 0.21 mL | 5.0 mL | Toluene | Ketone → Grignard | 68 | — | 26 | 7 |
| 006 | (5.30 eq.) | (0.67 eq.) | Cyclohexane | (0.5 mL) | over 20 min, −25° C. | | | | |
| | | | | and | Warmed to −5° C., 1 h | | | | |
| | | | | Cyclohexane | | | | | |
| | | | | (0.5 mL) | | | | | |
| AG- | 0.50 mL, | 0.21 mL | 5.0 mL | Toluene | Ketone → Grignard | 63 | — | 31 | 6 |
| 007 | (5.30 eq.) | (0.67 eq.) | Cyclohexane | (0.5 mL) | over 20 min, −25° C. | | | | |
| | | | | and | Warmed to 5° C., 4 h | | | | |
| | | | | Cyclohexane | | | | | |
| | | | | (0.5 mL) | | | | | |
| AG- | 0.50 mL, | 0.21 mL | 5.0 mL | Toluene | Ketone → Grignard | 70 | — | 25 | 5 |
| 008 | (5.30 eq.) | (0.67 eq.) | Cyclohexane | (0.5 mL) | over 20 min, −25° C. | | | | |
| | | | | and | Warmed to RT, | | | | |
| | | | | Cyclohexane | overnight | | | | |
| | | | | (0.5 mL) | | | | | |
| AG- | 0.50 mL, | 0.21 mL | 5.0 mL | Toluene | Ketone → Grignard | 57 | — | 21 | 22 |
| 009 | (5.30 eq.) | (0.67 eq.) | Toluene | (0.5 mL) | over 20 min, −25° C. | | | | |
| | | | | and | Warmed to RT, | | | | |
| | | | | Cyclohexane | overnight | | | | |
| | | | | (0.5 mL) | | | | | |
| AG- | 0.75 mL, | 0.25 mL, | 5,0 mL | Toluene | Ketone → Grignard | 78 | — | 15 | 7 |
| 010 | (8.0 eq.) | (0.80 eq.) | Cyclohexane | (0.5 mL) | over 20 min, −25° C. | | | | |
| | | | | and | Warmed to RT, | | | | |
| | | | | Cyclohexane | overnight | | | | |
| | | | | (0.5 mL) | | | | | |
| AG- | 1.00 mL, | 0.25 mL, | 5.0 mL | Toluene | Ketone → Grignard | 84 | — | 7 | 9 |
| 011 | (10.6 eq.) | (0.80 eq.) | Cyclohexane | (0.5 mL) | over 20 min, −15° C. | (65) | | | |
| | | | | and | Warmed to RT, | | | | |
| | | | | Cyclohexane | overnight | | | | |
| | | | | (0.5 mL) | | | | | |

^{a}Conversion analyzed by LCMS.
^{b}Diastereomer ratio determined by 1H NMR. Isolated yield (via column chromatography) given in parentheses.

TABLE 2
Overview of outcome of Grignard reaction using different reaction conditions. All reactions were cooled
to −5° C. before quenching with NH4Cl then extraction work-up with EtOAc, washing with brine.

| | Grignard Solution | | | | | Crude Product Mixture ^{a} | | | |
|---|---|---|---|---|---|---|---|---|---|
| | | LaCl₃•2LiCl | Diluent | | | | | % | |
| | t-BuMgCl | (0.6N | (Cyclohexane | | | % addition^{b} | | SM/ | % |
| entry | (2N in Et₂O) | in THF) | or Toluene) | Ketone Solution | Reaction Conditions | (S) | (R) | enol | reduced |
| AG- | 1.25 mL, | 0.25 mL | 5.0 mL | Toluene (0.5 mL) | Ketone → Grignard over 20 | 65 | — | 4 | 31 |
| 015 | (13.3 eq.) | (0.80 | Cyclohexane | and Cyclohexane | min, −15° C. | (65) | | | |
| | | eq.) | | (0.5 mL) | Warmed to RT, overnight | | | | |
| AG- | 1.00 mL, | 0.25 mL, | 5.0 mL | Toluene (0.5 mL) | Ketone → Grignard over 20 | 74 | — | 10 | 16 |
| 017 | (10.6 eq.) | (0.80 | Cyclohexane | and Cyclohexane | min, −40° C. | | | | |
| | | eq.) | | (0.5 mL) | Warmed to RT, overnight | | | | |
| AG- | 1.00 mL, | 0.21 mL, | 5.0 mL | Toluene (0.5 mL) | Ketone → Grignard over 20 | 75 | — | 8 | 17 |
| 018 | (10.6 eq.) | (0.67 | Cyclohexane | and Cyclohexane | min, −40° C. | | | | |
| | | eq.) | | (0.5 mL) | Warmed to RT, overnight | | | | |
| AG- | 1.00 mL, | 0.122 mL, | 5.0 mL | Toluene (0.5 mL) | Ketone → Grignard over 20 | 55 | — | 5 | 40 |
| 019 | (10.6 eq.) | (0.39 | Cyclohexane | and Cyclohexane | min, −40° C. | | | | |
| | | eq.) | | (0.5 mL) | Warmed to RT, overnight | | | | |
| AG- | 1.00 mL, | 0.25 mL, | 5.0 mL | and Cyclohexane | Ketone → Grignard over 20 | 84 | — | 5 | 11 |
| 020 | (10.6 eq.) | (0.80 | Cyclohexane | Toluene (0.5 mL) | min, RT, stirred overnight | (82) | | | |
| | | eq.) | | (0.5 mL) | | | | | |
| AG- | 1.00 mL, | 0.25 mL, | 5.0 mL | Toluene (0.5 mL) | Ketone → Grignard over 20 | 80 | — | 4 | 16 |
| 021 | (10.6 eq.) | (0.80 | Cyclohexane | and Cyclohexane | min, −15° C. | (79) | | | |
| | | eq.) | | (0.5 mL) | Warmed to RT, overnight | | | | |
| AG- | 1.00 mL, | 0.122 mL, | 5.0 mL | Toluene (0.5 mL) | Ketone → Grignard over 20 | >67 | — | 4 | <29 |
| 023 | (10.6 eq.) | (0.39 | Cyclohexane | and Cyclohexane | min, RT, stirred overnight | | | | |
| | | eq.) | | (0.5 mL) | | | | | |
| AG- | 1.12 mL, | 0.25 mL, | 5.0 mL | Toluene (0.5 mL) | Ketone → Grignard over 20 | 85 | — | 3 | 12 |
| 024 | (12.0 eq.) | (0.80 | Cyclohexane | and Cyclohexane | min, RT, stirred overnight | (84) | | | |
| | | eq.) | | (0.5 mL) | | | | | |

^{a} Conversion analyzed by LCMS.
^{b}Diastereomer ratio determined by 1H NMR. Isolated yield (via column chromatography) given in parentheses.

TABLE 3
Overview of outcome of Grignard reaction using different reaction conditions. All reactions were cooled
to −5° C. before quenching with NH4Cl then extraction work-up with EtOAc, washing with brine.

| | Grignard Solution | | | | | Crude Product Mixture^{a} | | | |
|---|---|---|---|---|---|---|---|---|---|
| | t-BuMgCl | | Diluent | | | | | % | |
| | (2N in | Salt/Lewis | (Cyclohexane | | | % addition^{b} | | SM/ | % |
| entry | Et₂O) | Acid/Additives* | or Toluene) | Ketone Solution | Reaction Conditions | (S) | (R) | enol | reduced |
| AG- | 1.12 mL, | 0.25 mL, 0.80 | 5.0 mL | Toluene (0.5 mL) | Ketone → Grignard over 20 | 85 | — | 3 | 12 |
| 024 | (12.0 eq.) | eq. | Cyclohexane | and | min, RT, stirred overnight | (84) | | | |
| LaCl₃ | | LaCl₃•2LiCl | | Cyclohexane | | | | | |
| | | (0.6N THF) | | (0.5 mL) | | | | | |
| AG- | 1.12 mL, | — | 5.0 mL | Toluene (0.5 mL) | Ketone → Grignard over 20 | 34 | — | 1 | 65 |
| 029 | (12.0 eq.) | | Cyclohexane | and | min, RT, stirred overnight | | | | |
| No | | | | Cyclohexane | | | | | |
| Salt | | | | (0.5 mL) | | | | | |
| AG- | 1.12 mL, | 0.25 mL, 0.80 | 2.5 mL | Toluene (0.5 mL) | Ketone → Grignard over 20 | 81 | — | 5 | 14 |
| 030 | (12.0 eq.) | eq. | Cyclohexane | and | min, RT, stirred overnight | | | | |
| Conc. | | LaCl₃•2LiCl | | Cyclohexane | | | | | |
| | | (0.6N THF) | | (0.5 mL) | | | | | |
| AG- | 1.12 mL, | 0.25 mL, 0.80 | 5.0 mL | Toluene (0.5 mL) | Ketone → Grignard over 20 | 82 | — | 3 | 15 |
| 031 | (12.0 eq.) | eq. | Cyclohexane | and | min, RT, stirred for 1 h | | | | |
| 1 h | | LaCl₃•2LiCl | | Cyclohexane | | | | | |
| | | (0.6N THF) | | (0.5 mL) | | | | | |
| AG- | 1.12 mL, | 92 mg, 2.00 | 5.0 mL | Toluene (0.5 mL) | Ketone → Grignard over 20 | 82 | — | 15 | 3 |
| 032 | (12.0 eq.) | eq. | Cyclohexane | and | min, RT, stirred overnight | | | | |
| CeCl₃ | | CeCl₃ | | Cyclohexane | | | | | |
| | | | | (0.5 mL) | | | | | |
| AG- | 1.12 mL, | 0.215 mL, | 0.5 mL | Toluene (0.25 | Ketone → Grignard over 20 | 90 | — | 5 | 5 |
| 034 | (12.0 eq.) | 8.00 eq. | Toluene | mL) and Diethyl | min, RT, stirred overnight | (88) | | | |
| Diglyme | | Diglyme | | Ether (0.25 mL) | | | | | |
| AG- | 1.12 mL, | 16 mg, 2.00 | 5.0 mL | Toluene (0.5 mL) | Ketone → Grignard over 20 | 73 | — | 18 | 9 |
| 038 | (12.0 eq.) | eq. | Cyclohexane | and | min, RT, stirred overnight | | | | |
| LiCl | | LiCl | | Cyclohexane | | | | | |
| | | | | (0.5 mL) | | | | | |
| AG- | 1.12 mL, | 51 mg, 2.00 | 5.0 mL | Toluene (0.5 mL) | Ketone → Grignard over 20 | 58 | — | 34 | 8 |
| 039 | (12.0 eq.) | eq. | Cyclohexane | and | min, RT, stirred overnight | | | | |
| ZnCl₂ | | ZnCl₂ | | Cyclohexane | | | | | |
| | | | | (0.5 mL) | | | | | |
| AG- | 1.12 mL, | 184 mg, 4.00 | 5.0 mL | Toluene (0.5 mL) | Ketone → Grignard over 20 | 76 | — | 20 | 4 |
| 040 | (12.0 eq.) | eq. | Cyclohexane | and | min, RT, stirred overnight | | | | |
| CeCl₃ | | CeCl₃ | | Cyclohexane | | | | | |
| | | | | (0.5 mL) | | | | | |

^{a}Conversion analyzed by LCMS.
^{b}Diastereomer ratio determined by 1H NMR. Isolated yield (via column chromatography) given in parentheses.

TABLE 4
Overview of outcome of Grignard reaction using different reaction conditions. Isolated yield (via column chromatography) given in parentheses.

| | Grignard Solution | | | | | Crude Product Mixture^{a} | | | |
|---|---|---|---|---|---|---|---|---|---|
| | t-BuMgCl | | Diluent | | | | | % | % |
| | (2N in | Salt/Lewis | (Cyclohexane | | | % addition^{b} | | SM/ | reduce |
| entry | Et₂O) | Acid/Additives* | or Toluene) | Ketone Solution | Reaction Conditions | (S) | (R) | enol | d |
| AG- | 1.12 mL, | 0.215 mL, 8.00 | 0.5 mL | Toluene (0.25 mL) | Ketone → Grignard over 20 min, | 90 | — | 5 | 5 |
| 034 | (12.0 eq.) | eq. | Toluene | and Diethyl Ether | RT, stirred overnight | (88) | | | |
| | | Diglyme added | | (0.25 mL) | | | | | |
| | | at 0° C. | | | | | | | |
| AG- | 1.12 mL, | 0.215 mL, 8.00 | 0.5 mL | Toluene (0.25 mL) | Ketone → Grignard over 20 min, | 91 | — | 3 | 6 |
| 043 | (12.0 eq.) | eq. | Toluene | and Cyclohexane | RT, stirred overnight | | | | |
| | | Diglyme added | | (0.25 mL) | | | | | |
| | | at 0° C. | | | | | | | |
| AG- | 1.12 mL, | 0.215 mL, 8.00 | 0.5 mL | Toluene (0.25 mL) | Ketone → Grignard over 20 min, | 41 | — | 52 | 7 |
| 044 | (12.0 eq.) | eq. | Toluene | and Diglyme (0.25 | RT, stirred overnight | | | | |
| | | Diglyme added | | mL) | | | | | |
| | | at 0° C. | | | | | | | |
| AG- | 1.12 mL, | 0.215 mL, 8.00 | 0.5 mL | Toluene (0.5 mL) | Ketone → Grignard over 20 min, | 90 | — | 3 | 7 |
| 045 | (12.0 eq.) | eq. | Toluene | | RT, stirred overnight | | | | |
| | | Diglyme added | | | | | | | |
| | | at 0° C. | | | | | | | |
| AG- | 1.12 mL, | 0.322 mL, 12.0 | 0.5 mL | Toluene (0.25 mL) | Ketone → Grignard over 20 min, | 64 | — | 29 | 7 |
| 046 | (12.0 eq.) | eq. | Toluene | and Diethyl Ether | RT, stirred overnight | | | | |
| | | Diglyme added | | (0.25 mL) | | | | | |
| | | at 0° C. | | | | | | | |
| AG- | 1.12 mL, | 0.215 mL, 8.00 | 0.5 mL | Toluene (0.25 mL) | Ketone → Grignard over 20 min, | 71 | — | 17 | 12 |
| 047 | (12.0 eq.) | eq. | Toluene | and Diethyl Ether | 0° C. | | | | |
| | | Diglyme added | | (0.25 mL) | Warmed to RT, overnight | | | | |
| | | at 0° C. | | | | | | | |
| AG- | 1.12 mL, | 0.215 mL, 8.00 | 0.5 mL | Toluene (0.25 mL) | Ketone → Grignard over 20 min, | 90 | — | 5 | 5 |
| 048 | (12.0 eq.) | eq. | Toluene | and Diethyl Ether | RT, stirred for 1 h | | | | |
| | | Diglyme added | | (0.25 mL) | | | | | |
| | | at 0° C. | | | | | | | |
| AG- | 0.56 mL, | 0.108 mL, 4.00 | 0.5 mL | Toluene (0.25 mL) | Ketone → Grignard over 20 min, | 95 | — | 2 | 3 |
| 053 | (6.0 eq.) | eq. | Toluene | and Diethyl Ether | RT, stirred overnight | (92) | | | |
| | | Diglyme added | | (0.25 mL) | | | | | |
| | | at 0° C. | | | | | | | |
| AG- | 0.56 mL, | 0.108 mL, 4.00 | 0.5 mL | Toluene (0.25 mL) | Ketone → Grignard over 20 min, | 94 | — | 2 | 4 |
| 054 | (6.0 eq.) | eq. | Cyclohexane | and Diethyl Ether | RT, stirred overnight | Solubility issues | | | |
| | | Diglyme added | | (0.25 mL) | | (initial clumps) | | | |
| | | at 0° C. | | | | | | | |
| AG- | 0.56 mL, | 0.054 mL, 2.00 | 0.5 mL | Toluene (0.25 mL) | Ketone → Grignard over 20 min, | 76 | — | 3 | 21 |
| 057 | (6.0 eq.) | eq. | Toluene | and Cyclohexane | RT, stirred overnight | Solubility issues | | | |
| | | Diglyme added | | (0.25 mL) | | (clumps after ketone) | | | |
| | | at 0° C. | | | | | | | |
| AG- | 0.56 mL, | 0.054 mL, 2.00 | 0.5 mL | Toluene (0.25 mL) | Ketone → Grignard over 20 min, | 76 | — | 4 | 20 |
| 058 | (6.0 eq.) | eq. | Toluene | and Diethyl Ether | RT, stirred overnight | | | | |
| | | Diglyme added | | (0.25 mL) | | | | | |
| | | at 0° C. | | | | | | | |
| AG- | 0.281 mL, | 0.054 mL, 2.00 | 0.5 mL | Toluene (0.25 mL) | Ketone → Grignard over 20 min, | 80 | — | 14 | 6 |
| 059 | (3.0 eq.) | eq. | Toluene | and Diethyl Ether | RT, stirred overnight | | | | |
| | | Diglyme added | | (0.25 mL) | | | | | |
| | | at 0° C. | | | | | | | |
| AG- | 0.140 mL, | 0,040 mL, 1.50 | 0.5 mL | Toluene (0.25 mL) | Ketone → Grignard over 20 min, | 52 | — | 43 | 5 |
| 060 | (1.5 eq.) | eq. | Toluene | and Diethyl Ether | RT, stirred overnight | | | | |
| | | Diglyme added | | (0.25 mL) | | | | | |
| | | at 0° C. | | | | | | | |

TABLE 5
Overview of outcome of Grignard reaction using different reaction conditions.

| | Grignard Solution | | | | | Crude Product Mixture^{a} | | | |
|---|---|---|---|---|---|---|---|---|---|
| | t- | | | | | | | | |
| | BuMgCl | | Diluent | | | | | % | |
| | (2N in | Salt/Lewis | (Cyclohexane | | | % addition^{b} | | SM/ | % |
| entry | Et₂O) | Acid/Additives* | or Toluene) | Ketone Solution | Reaction Conditions | (S) | (R) | enol | reduced |
| AG- | 1.12 mL, | 0.215 mL, | 312 mg, 6.00 | Toluene (0.25 | Ketone → Grignard over 20 | 53 | — | 36 | 11 |
| 049 | (12.0 eq.) | 8.00 eq. | eq. NBu₄Cl | mL) and Diethyl | min, RT, stirred overnight | | | | |
| | | Diglyme | | Ether (0.25 mL) | | | | | |
| | | added at 0° C. | | | | | | | |
| AG- | 1.12 mL, | 0.215 mL, | 52 mg, 1.0 | Toluene (0.25 | Ketone → Grignard over 20 | 74 | — | 5 | 21 |
| 050 | (12.0 eq.) | 8.00 eq. | eq. NBu₄Cl | mL) and Diethyl | min, RT, stirred overnight | | | | |
| | | Diglyme | | Ether (0.25 mL) | | | | | |
| | | added at 0° C. | | | | | | | |
| AG- | 0.56 mL, | 0.108 mL, | 52 mg, 1.0 | Toluene (0.25 | Ketone → Grignard over 20 | 79 | — | 6 | 15 |
| 051 | (6.0 eq.) | 4.0 eq. | eq. NBu₄Cl | mL) and Diethyl | min, RT, stirred overnight | | | | |
| | | Diglyme | | Ether (0.25 mL) | | | | | |
| | | added at 0° C. | | | | | | | |
| AG- | 0.56 mL, | 0.108 mL, | 52 mg, 1.0 | Toluene (0.25 | Ketone → Grignard over 20 | 47 | — | 36 | 17 |

| | | | | | | |
|---|---|---|---|---|---|---|
| 052 | (6.0 eq.) | 4.0 eq. | eq. | mL) and Diethyl | min, RT, stirred overnight | Immiscible gum |
| | | Diglyme | NBu₄Cl/0.5 | Ether (0.25 mL) | | and clear solvent |

| | |
|---|---|
| added at 0° C. | ml Toluene |

TABLE 6
Overview of outcome of Grignard reaction using different reaction conditions.

| | Grignard Solution | | | | | Crude Product Mixture^{a} | | | |
|---|---|---|---|---|---|---|---|---|---|
| | t- | | | | | | | | |
| | BuMgCl | Salt/Lewis | Diluent | | | | | % | |
| | (2N in | Acid/Additives* | (Cyclohexane | Ketone | Reaction | % addition^{b} | | SM/ | % |
| entry | Et₂O) | Diglyme added at 0° C. | or Toluene) | Solution | Conditions | (S) | (R) | enol | reduced |
| AG- | 1.12 mL | 0.25 mL, 0.80 eq. | 0.5 mL | Toluene (0.25 | Ketone → | 71 | — | 25 | 4 |
| 055 | (12.0 eq.) | LaCl₃•2LiCl (0.6N THF) | Toluene | mL) and | Grignard over 20 | | | | |
| | | 0.215 mL, 8.00 eq. | | Cyclohexane | min, RT, stirred | | | | |
| | | Diglyme | | (0.25 mL) | for 1 h | | | | |
| AG- | 1.12 mL | 92 mg, 2.00 eq. CeCl₃ | 0.5 mL THF | Toluene (0.25 | Ketone → | 52 | — | 41 | 7 |
| 056 | (12.0 eq.) | 0.215 mL, 8.00 eq. | and 0.5 mL | mL) and | Grignard over 20 | | | | |
| | | Diglyme | Cyclohexane | Cyclohexane | min, RT, stirred | | | | |
| | | | | (0.25 mL) | overnight | | | | |

TABLE 7
Overview of outcome of Grignard reaction using different reaction conditions.

| | | | | | | | Crude Product | | |
|---|---|---|---|---|---|---|---|---|---|
| | Grignard Solution | | | | | | Mixture^{a} | | |
| | t-BuMgCl | | Diluent | Ketone | | | % | % | |
| | (2N in | Salt/Lewis | (Cyclohexane | (Purification | | | addition^{b} | SM/ | % |
| entry | Et₂O) | Acid/Additives* | or Toluene) | Method) | Ketone Solution | Reaction Conditions | (S) | enol | reduced |
| AG- | 1.12 mL, | 0.108 mL, | 0.5 mL | Column | Toluene (0.25 mL) | Ketone → Grignard | Bottle | Abandoned | |
| 067 | (6.0 eq.) | 4.00 eq. | Toluene | | and Diethyl Ether | over 20 min, RT, | Lid | | |
| | | Triglyme | | | (0.25 mL) | stirred overnight | Fused | | |
| | | added at 0° C. | | | | | (1969) | | |
| AG- | 1.12 mL, | 0.108 mL, | 0.5 mL | Column | Toluene (0.25 mL) | Ketone → Grignard | 57 | 40 | 3 |
| 068 | (6.0 eq.) | 4.00 eq. | Toluene | | and Diethyl Ether | over 20 min, RT, | | | |
| | Ether | 12-crown-4 at | | | (0.25 mL) | stirred overnight | | | |
| | added | 0° C. | | | | | | | |
| AG- | 1.12 mL, | 0.108 mL, | 0.5 mL | Column | Toluene (0.25 mL) | Ketone → Grignard | 65 | 33 | 2 |
| 075 | (6.0 eq.) | 4.00 eq. | Toluene | | and Diethyl Ether | over 20 min, | | | |
| | Ether | 15-crown-5 at | | | (0.25 mL) | RT, stirred overnight | | | |
| | added | 0° C. | | | | | | | |
| AG- | 1.12 mL, | 0.108 mL, | 0.5 mL | Column | Toluene (0.25 mL) | Ketone → Grignard | 62 | 35 | 2 |
| 076 | (6.0 eq.) | 4.00 eq. | Toluene | | and Diethyl Ether | over 20 min, | | | |
| | Added to | 15-crown-5 at | | | (0.25 mL) | RT, stirred overnight | | | |
| | Ether | 0° C. | | | | | | | |
| AG- | 1.12 mL, | 0.108 mL, | 0.5 mL | Column | Toluene (0.25 mL) | Ketone → Grignard | 92 | 6 | 2 |
| 069 | (6.0 eq.) | 4.00 eq. | Toluene | | and Diethyl Ether | over 20 min, RT, | | | |
| | Added to | 18-crown-6 at | | | (0.25 mL) | stirred overnight | | | |
| | Ether | 0° C. | | | | | | | |
| AG- | 1.12 mL, | 0.108 mL, | 0.5 mL | Column | Toluene (0.25 mL) | Ketone → Grignard | Ordered | Abandoned | |
| 070 | (6.0 eq.) | 4.00 eq. | Toluene | | and Diethyl Ether | over 20 min, RT, | DGDE | | |
| | | DGDE added | | | (0.25 mL) | stirred overnight | | | |
| | | at 0° C. | | | | | | | |

### Conclusions

The present example demonstrates that a ratio of 6:4 between the Grignard reagent and the additive, diglyme was beneficial for suppressing side-reactions and increasing the yield. The present example further demonstrates that the use of an ethereal solvent in combination with an additive, such as a glyme e.g. diglyme or a crown ether, such as 18-crown-6 increased the yield of the desired product.

### Example 2—Grignard Reaction with Diglyme and Diethyl Ether/Toluene

To a 20 ml microwave vial containing tert-butylmagnesium chloride in diethyl ether (2 M, 0.560 mL, 6.0 eq.) in anhydrous Toluene (0.5 mL) was added diglyme (0.108 mL, 4 eq.) dropwise at 0° C. and the mixture was allowed to stir for 1 h at 0° C. before being warmed to RT. The ketone, BnO-II-Bn (100 mg) was dissolved in a separate 20 mL microwave vial and dissolved in 0.50 ml of a mixture of toluene and diethyl ether (1:1) and added to the Grignard solution over 20 min at RT and the mixture was allowed to stir overnight. (Each drop when added becomes bright yellow and fades immediately). (Reaction doesn't need overnight, 1-2 h is sufficient). The reaction mixture was cooled to −5 C before being quenched with 4 M NH4Cl and subjected to an extraction work-up with ethyl acetate (3×25 mL), washed with brine, dried over MgSO₄ and concentrated under reduced pressure to provide BnO-III-Bn (101.5 mg, 92%).

### Conclusion

The present example demonstrates that performing the Grignard reaction with diglyme and, diethyl ether and toluene provides a surprisingly high yield of the desired stereoisomer, BnO-IIIA-Bn and a high purity profile.

### Example 3—Grignard Reaction Using Solvent Swap Conditions

To a 20 mL microwave vial containing tert-butylmagnesium chloride in diethyl ether (2 M, 0.560 mL, 6.0 eq.) was added a vacuum line and the solvent was evaporated until dry. CPME (0.560 mL) was then added to the dried Grignard solid to provide a 2 N solution. To this vial was added anhydrous Toluene (0.5 mL) and diglyme (0.108 mL, 4 eq.) dropwise and the mixture was allowed to stir for 1 h at 0° C. and warmed to RT. The ketone, BnO-II-Bn (100 mg) was dissolved in a separate 20 mL microwave vial and dissolved in 0.50 ml of toluene and added to the Grignard solution over 20 min at RT and the mixture was allowed to stir overnight. The reaction mixture was cooled to −5 C before being quenched with 4 M NH₄Cl and subjected to an extraction work-up with ethyl acetate (3×25 mL), washed with brine, dried over MgSO₄ and concentrated under reduced pressure. Analysis of the resulting mixture by NMR/LCMS showed 91% product, 2% starting material and 7% reduced side product.

In table 8 below, a series of reactions with different solvents were carried out using procedures as in Example 3. The solvent referred to under “Solvent Swap” was added to the dried Grignard solvent, exemplified above using CPME.

TABLE 8
overview of results from solvent swap experiments.

| | Grignard Solution | | | | | | | Crude Product | | |
|---|---|---|---|---|---|---|---|---|---|---|
| | t- | | | | | | | Mixture^{a} | | |
| | BuMgCl | Salt/Lewis | | | | | | % | % | |
| | (2N in | Acid/ | | Solvent | Dielectric | Ketone | | addition^{b} | SM/ | % |
| entry | Et₂O) | Additives* | Diluent | Swap | constant (E) | Solution | Reaction Conditions | (S) | enol | reduced |
| AG- | 0.56 mL, | 0.108 mL, | 0.5 mL | 1,4- | 2.25 | Toluene | Ketone → Grignard over | 58 | 38 | 4 |
| 095 | (6.0 eq.) | 4.00 eq. | Toluene | Dioxane | | (0.5 mL) | 20 min, RT, stirred | | | |
| | | Diglyme added | | | | | overnight | | | |
| | | at 0° C. | | | | | | | | |
| AG- | 0.56 mL, | — | 0.5 mL | 1,4- | 2.25 | Toluene | Ketone → Grignard over | 73 | 22 | 5 |
| 104 | (6.0 eq.) | | Toluene | Dioxane | | (0.5 mL) | 20 min, RT, stirred | | | |
| | | | | | | | overnight | | | |
| AG- | 1.12 mL, | — | 0.5 mL | 1,4- | 2.25 | Toluene | Ketone → Grignard over | 57 | 39 | 4 |
| 105 | (12.0 eq.) | | Toluene | Dioxane | | (0.5 mL) | 20 min, RT, stirred | | | |
| | | | | | | | overnight | | | |
| AG- | 0.56 mL, | 0.108 mL, | 0.5 mL | Toluene | 2.38 | Toluene | Ketone → Grignard | 50 | 42 | 8 |
| 086 | (6.0 eq.) | 4.00 eq. | Toluene | | | (0.5 mL) | over 20 min, | | | |
| | | Diglyme added | | | | | RT, stirred overnight | | | |
| | | at 0° C. | | | | | | | | |
| AG- | 0.56 mL, | 0.108 mL, | 0.5 mL | Diethyl | 4.34 | Toluene | Ketone → Grignard over | 90 | 2 | 8 |
| 090 | (6.0 eq.) | 4.00 eq. | Toluene | Ether | | (0.5 mL) | 20 min, RT, stirred | | | |
| | | Diglyme added | | | | | overnight | | | |
| | | at 0° C. | | | | | | | | |
| AG- | 0.56 mL, | 0.108 mL, | 0.5 mL | MTBE | 4.5 | Toluene | Ketone → Grignard over | 90 | 2 | 8 |
| 117 | (6.0 eq.) | 4.00 eq. | Toluene | | | (0.5 mL) | 20 min, RT, stirred | | | |
| | | Diglyme added | | | | | overnight | | | |
| | | at 0° C. | | | | | | | | |
| AG- | 0.56 mL, | 0.108 mL, | 0.5 mL | CPME | 4.8 | Toluene | Ketone → Grignard over | 86 | 6 | 8 |
| 094 | (6.0 eq.) | 4.00 eq. | Toluene | | | (0.5 mL) | 20 min, RT, stirred | | | |
| | | Diglyme added | | | | | overnight | | | |
| | | at 0° C. | | | | | | | | |
| AG- | 0.56 mL, | 0.108 mL, | 0.5 mL | CPME | 4.8 | Toluene | Ketone → Grignard over | 91 | 2 | 7 |
| 103 | (6.0 eq.) | 4.00 eq. | Toluene | | | (0.5 mL) | 20 min, RT, stirred | | | |
| | | Diglyme added | | | | | overnight | | | |
| | | at 0° C. | | | | | | | | |
| AG- | 0.56 mL, | 0.108 mL, | 0.5 mL | DME | 5.0 | Toluene | Ketone → Grignard over | 60 | 13 | 27 |
| 098 | (6.0 eq.) | 4.00 eq. | Toluene | | | (0.5 mL) | 20 min, RT, stirred | | | |
| | | Diglyme added | | | | | overnight | | | |
| | | at 0° C. | | | | | | | | |
| AG- | 0.56 mL, | 0.108 mL, | 0.5 mL | 2- | 7.0 | Toluene:MeTHF | Ketone → Grignard over | 55* | 26 | 19 |
| 072 | (6.0 eq.) | 4.00 eq. | Toluene | MeTHF | | (0.5 mL) | 20 min, RT, stirred | | | |
| | | Diglyme added | | | | | overnight | | | |
| | | at 0° C. | | | | | | | | |
| AG- | 0.56 mL, | 0.108 mL, | 0.5 mL | THF | 7.6 | Toluene | Ketone → Grignard over | 35 | 53 | 12 |
| 119 | (6.0 eq.) | 4.00 eq. | Toluene | | | (0.5 mL) | 20 min, RT, stirred | | | |
| | | Diglyme added | | | | | overnight | | | |
| | | at 0° C. | | | | | | | | |

### Conclusion

The present example demonstrates that varying the solvent system significantly influences the yield and the purity profile of the resulting product mixture. Preparing the Grignard in the process chemistry suitable solvent, CPME with the additive glyme affords the desired product in very high yield.

### Example 4—Grignard Reaction Using Scale-Up Conditions

To a reaction container was added tBuMgCl (224.5 mmol, 8 eq., 0.25M solution in CPME) followed by toluene (75 mL) and diglyme (150 mmol, 5.33 eq.) under stirring. Subsequently, BnO-II-Bn (28.1 mmol, in toluene 20.4% wt, 1 eq.) was added at 10° C. and the temperature was adjusted to 20-30° C. After 3 h, the reaction mixture was cooled to approximately 5° C. and NH₄Cl (aq, 5%, 140 mmol, 5 eq.) was added. The temperature was maintained below 20° C. The mixture was passed to a second container via filter pad and the mixture stirred, settled, and separated. Water (300 mL) was added to the remaining organic phase, the resulting phases stirred, settled and separated. The organic phase was concentrated in vacuo. To the concentrated material, ethanol (300 mL) was added. The resulting solution was concentrated in vacuo. To the concentrated material, more ethanol (300 mL) was added. The resulting solution was concentrated in vacuo to a concentrated solution. Ethanol (75 mL) was added to the concentrated solution and the resulting suspension was heated to reflux (78° C.) for 30 minutes and subsequently cooled to 50° C. for 30 minutes and stirred at 50° C. for 1 hour. The contents were further cooled to 5° C. over 1 hour and stirred at 5° C. for 2 hours. The solid material was collected, washed with cold (5° C.) ethanol, and dried in vacuo at 40-45° C. to provide the desired in product in 66% yield, 99% purity.

### Example 5—Isolation Procedure

A crude mixture of compound Bn-O-IIIA-Bn was obtained essentially as described in Example 4 up until and including addition of NH₄Cl and removal of undesired solids by filtration. The crude mixture was diluted with ethanol, the mixture agitated at 200 rpm and the temperature increased to 78° C. The mixture was stirred at 78° C. for 30 minutes and subsequently cooled to 45-55° C. over the course of 30 minutes. The mixture was further stirred at 45-55° C. for 1 hour and subsequently cooled to 5° C. over the course of 1 hour. The mixture was further stirred for 2 hours, the mixture passed through a filter, and ethanol was added to the resulting filtrate. The solids were collected by filtration and dried in vacuo to provide the desired product in 86% yield.

### Items of the Invention

Further described herein are the following itemized embodiments:
1. A method of preparing a compound of formula Pg-O-IIIA-Pg*,

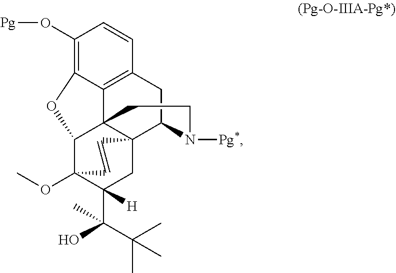

comprising mixing a compound of formula Pg-O-II-Pg*,

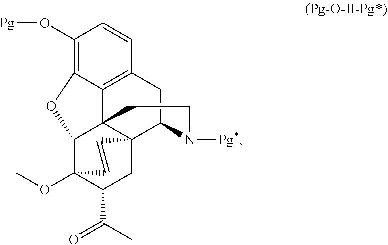

with a tert-butylmagnesium compound and one or more additives in a solvent system to provide the compound of formula Pg-O-IIIA-Pg*; wherein each Pg and Pg* is a hydroxy-protecting group and Pg and Pg* the same or different.
2. The compound according to item 1, wherein each Pg and Pg* is selected from the group consisting of: benzyl (Bn), methoxymethyl (MOM), tetrahydropyranyl (THP), tert-butyl (tBu), allyl, tert-butyldimethylsilyl (TBDMS), tert-butyldiphenylsilyl (TBDPS), acetyl (Ac), pivaloyl (Piv), and benzoyl (Bz).
3. The method according to any one of the preceding items, wherein Pg and Pg* are benzyl (Bn), such that the compound of formula Pg-O-IIIA-Pg* is compound BnO-IIIA-Bn,

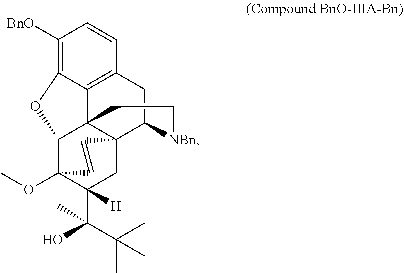 [SMILES: CO[C@]12C=C[C@@]3(C[C@@H]1[C@](C)(O)C(C)(C)C)[C@H]1Cc4ccc(OCc5ccccc5)c5c4[C@@]3(CCN1Cc1ccccc1)[C@H]2O5]

and the compound of formula Pg-O-II-Pg* is compound BnO-II-Bn,

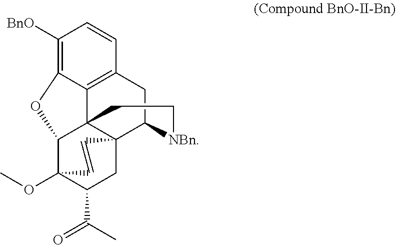 [SMILES: CO[C@]12C=C[C@@]3(C[C@@H]1C(C)=O)[C@H]1Cc4ccc(OCc5ccccc5)c5c4[C@@]3(CCN1Cc1ccccc1)[C@H]2O5]

4. The method according to any one of the preceding items, wherein the method comprises use of one additive.
5. The method according to any one of the preceding items, wherein the one or more additives are selected from the group consisting of: a non-cyclic polyether, a cyclic polyether, a Lewis acid, and a combination thereof.
6. The method according to any one of the preceding items, wherein the non-cyclic polyether is a glyme.
7. The method according to any of the preceding items, wherein the glyme is selected from the group consisting of: monoglyme, diglyme, triglyme, tetraglyme, ethyl glyme, ethyl diglyme, butyl glyme, butyl diglyme, and polyglyme.
8. The method according to any one of the preceding items, wherein the glyme is diglyme.
9. The method according to any one of the preceding items, wherein the one or more additives are added to the solvent system in a stochiometry with respect to the compound Pg-O-II-Pg* of from 1 equivalent to 8 equivalents, such as from 2 equivalents to 7 equivalents, such as from 3 equivalents to 6 equivalents, for example 4 equivalents.
10. The method according to any one of the preceding items, wherein the method comprises the steps:
   a. adding the tert-butylmagnesium compound to a first solvent system to provide a solution of the tert-butylmagnesium compound;
   b. adding Compound Pg-O-II-Pg* to a second solvent system to provide a solution of Pg-O-II-Pg*; and
   c. adding the solution of Pg-O-II-Pg* over a time period to the solution of the tert-butylmagnesium compound; or adding the solution of the tert-butylmagnesium compound over a time period to the solution of Pg-O-II-Pg*.
11. The method according to any one of the preceding items, wherein the time period is from 10 to 80 minutes per mL of solution of BnO-II-Bn, for example 20 minutes for 0.5 mL solution of BnO-II-Bn.
12. The method according to any one of the preceding items, wherein the time period is at least 1 minute, such as at least 2 minutes, such as at least 5 minutes, such as at least 10 minutes, such as at least 20 minutes, such as at least 40 minutes, such as at least 60 minutes, such as at least 80 minutes, such as at least 100 minutes, such as at least 120 minutes.
13. The method according to any one of the preceding items, wherein the time period is 6 hours or less, such as 5 hours or less, such as 4 hours or less, such as 3 hours or less.
14. The method according to any one of the preceding items, wherein BnO-II-Bn and the tert-butylmagnesium compound are mixed and allowed to react for at least 1 hour after mixing, such as from 1 hour to 24 hours after mixing.
15. The method according to any one of the preceding items, wherein BnO-II-Bn and the tert-butylmagnesium compound are mixed at a temperature within the range of from 15° C. to 100° C., such as from 20° C. to 100° C., such as from 25° C. to 100° C., such as from 30° C. to 100° C., such as from 15° C. to 95° C., such as from 15° C. to 90° C., such as from 15° C. to 85° C., such as from 20° C. to 95° C., such as from 25° C. to 90° C.
16. The method according to any one of the preceding items, wherein BnO-II-Bn and the tert-butylmagnesium compound are mixed at a temperature within the range of from 15° C. to 100° C., such as from 15° C. to 90° C., such as from 15° C. to 80° C., such as from 15° C. to 70° C., such as from 15° C. to 60° C.
17. The method according to any one of the preceding items, wherein the tert-butylmagnesium compound is selected from the group consisting of: a tert-butylmagnesium halide and di-tert-butylmagnesium.
18. The method according to any one of the preceding items, wherein the tert-butylmagnesium compound is tert-butylmagnesium chloride.
19. The method according to any one of the preceding items, wherein the tert-butylmagnesium compound is added to the solvent system in a stochiometry with respect to the compound BnO-II-Bn of from 2 equivalents to 12 equivalents, such as from 2 equivalents to 11 equivalents, such as from 2 equivalents to 10 equivalents, such as from 3 equivalents to 9 equivalents, such as from 4 equivalents to 8 equivalents, such as from 5 equivalents to 9 equivalents, such as from 6 to 7 equivalents, for example 6 equivalents.
20. The method according to any one of the preceding items, wherein the mol ratio of the tert-butylmagnesium compound is at least 6:4 and at most 12:4 with respect to the mol sum of one or more additives, optionally wherein the mol ratio is with respect to BnO-II-Bn as the limiting factor, optionally wherein one additive is used, such as glyme.
21. The method according to any one of the preceding items, wherein the solvent system, the first solvent system, and/or the second solvent system comprises a solvent having a dielectric constant of 7 or less, such as 6 or less, such as 5 or less.
22. The method according to any one of the preceding items, wherein the solvent system, the first solvent system, and/or the second solvent system comprises a solvent having a dielectric constant of from 1 to 6, such as from 1 to 5.
23. The method according to any one of the preceding items, wherein the solvent system, the first solvent system, and/or the second solvent system comprises an ethereal solvent.
24. The method according to any one of items 19-21, wherein the ethereal solvent has a dielectric constant of 7 or less, such as 6 or less, such as 5 or less.
25. The method according to any one of items 19-21, wherein the ethereal solvent has a dielectric constant of from 1 to 6, such as from 1 to 5.
26. The method according to any one of the preceding items, wherein the ethereal solvent is selected from the group consisting of: diethyl ether, cyclopentyl methyl ether (CPME), tert-butyl methyl ether (MTBE) and tetrahydrofurane (THF).
27. The method according to any one of the preceding items, wherein the first solvent system comprises a nonpolar solvent.
28. The method according to any one of the preceding items, wherein the nonpolar solvent of the first solvent system is toluene.
29. The method according to any one of the preceding items, wherein the first solvent system comprises a nonpolar solvent and an ethereal solvent.
30. The method according to any one of the preceding items, wherein the first solvent system comprises a nonpolar solvent and an ethereal solvent in a ratio of 20:80 by volume to 80:20 by volume such as 30:70, such as 40:60, such as 50:50, such as 60:40, such as 70:30, such as 80:20.
31. The method according to any one of the preceding items, wherein the second solvent system comprises a nonpolar solvent.
32. The method according to any one of the preceding items, wherein the nonpolar solvent of the second solvent system is toluene.
33. The method according to any one of the preceding items, wherein the method comprises mixing Compound BnO-II-Bn with tert-butylmagnesium chloride and diglyme in a solvent system at a temperature within the range of from 15° C. to 60° C. and allowed to react for at least 1 hour after mixing to provide Compound BnO-IIIA-Bn.
34. The method according to item 33, wherein diglyme is added to the solvent system in a stochiometry with respect to the compound BnO-II-Bn of from 1 equivalent to 6 equivalents, such as from 2 equivalents to 6 equivalents, such as 4 equivalents.
35. The method according to any of items 27-28, wherein the solvent system comprises a) an ethereal solvent, such as diethyl ether or cyclopentyl methyl ether, and b) a nonpolar solvent, such as toluene.
36. The method according to any of items 27-29, wherein a) the tert-butylmagnesium chloride and diglyme is mixed in an ethereal solvent and optionally a non-polar solvent; and Compound BnO-II-Bn is added to a nonpolar solvent, such as toluene, and optionally also an ethereal solvent and then b) the compound BnO-II-Bn in the nonpolar solvent is added to the ethereal solvent of a).
37. A method for preparing buprenorphine, or a salt thereof, comprising the method as defined in any preceding items.
38. The method according to any preceding items, wherein the method further comprises one, more or all of the steps of:
   step (i)(F): in a solvent system S1 comprising a polar protic solvent, reacting Compound HO-I-H

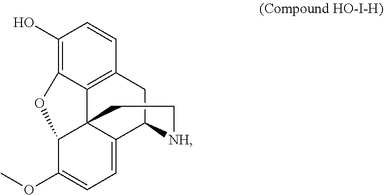 [SMILES: COC1=CC=C2[C@H]3Cc4ccc(O)c5c4[C@@]2(CCN3)[C@H]1O5]

with benzyl halide, benzyl sulfonate, or activated benzyl alcohol to provide Compound BnO-I-Bn:

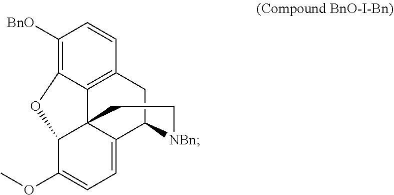 [SMILES: COC1=CC=C2[C@H]3Cc4ccc(OCc5ccccc5)c5c4[C@@]2(CCN3Cc2ccccc2)[C@H]1O5]

step (ii)(B): in a solvent system S2 comprising a polar protic solvent, reacting Compound BnO-I-Bn with methyl vinyl ketone to provide Compound BnO-II-Bn:

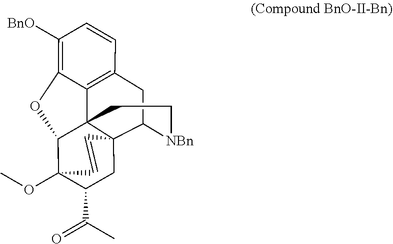 [SMILES: CO[C@]12C=C[C@@]3(C[C@@H]1C(C)=O)C1Cc4ccc(OCc5ccccc5)c5c4[C@@]3(CCN1Cc1ccccc1)[C@H]2O5]

step (iii)(D): in a solvent system S3 comprising a nonpolar solvent, reacting Compound BnO-II-Bn with a tert-butylmagnesium compound to provide Compound BnO-IIIA-Bn:

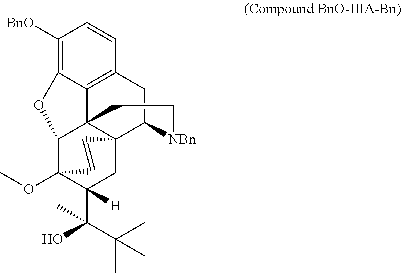 [SMILES: CO[C@]12C=C[C@@]3(C[C@@H]1[C@](C)(O)C(C)(C)C)[C@H]1Cc4ccc(OCc5ccccc5)c5c4[C@@]3(CCN1Cc1ccccc1)[C@H]2O5]

Step (iv)(C): reacting Compound BnO-IIIA-Bn with H₂ in the presence of a hydrogenation catalyst to provide a compound of Compound HO-IV-H:

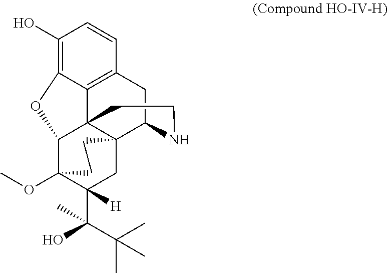 [SMILES: CO[C@]12CC[C@@]3(C[C@@H]1[C@](C)(O)C(C)(C)C)[C@H]1Cc4ccc(O)c5c4[C@@]3(CCN1)[C@H]2O5]

Step (v)(A1): reacting Compound HO-IV-H with cyclopropane carboxaldehyde followed by a hydride source; or
Step (v)(A2): reacting Compound HO-IV-H with cyclopropanecarboxylic acid halide followed by a reducing agent; or
Step (v)(A3): reacting Compound HO-IV-H with cyclopropylmethyl halide or activated cyclopropane methanol;
to provide buprenorphine.

1. A method of preparing a compound of formula Pg-O-IIIA-Pg*, 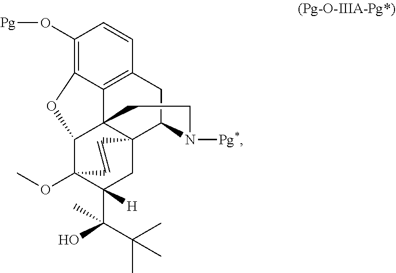
comprising mixing a compound of formula Pg-O-II-Pg*, 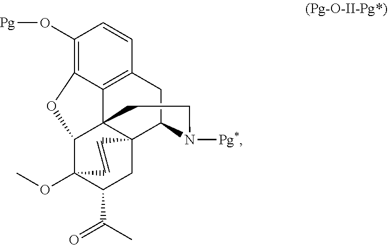
with a tert-butylmagnesium compound and one or more additives in a solvent system to provide the compound of formula Pg-O-IIIA-Pg*; wherein each Pg and Pg* is a hydroxy-protecting group and Pg and Pg* the same or different; wherein each Pg and Pg* is selected from the group consisting of: benzyl (Bn), methoxymethyl (MOM), tetrahydropyranyl (THP), tert-butyl (tBu), allyl, tert-butyldimethylsilyl (TBDMS), tert-butyldiphenylsilyl (TBDPS), acetyl (Ac), pivaloyl (Piv), and benzoyl (Bz).

2. The method according to claim 1, wherein Pg and Pg* are benzyl (Bn), such that the compound of formula Pg-O-IIIA-Pg* is compound BnO-IIIA-Bn, 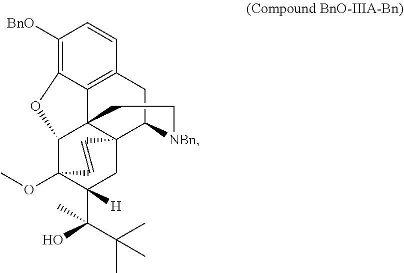 [SMILES: CO[C@]12C=C[C@@]3(C[C@@H]1[C@](C)(O)C(C)(C)C)[C@H]1Cc4ccc(OCc5ccccc5)c5c4[C@@]3(CCN1Cc1ccccc1)[C@H]2O5]
and the compound of formula Pg-O-II-Pg* is compound BnO-II-Bn,

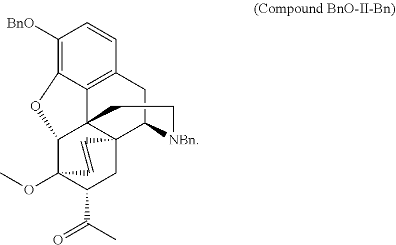 [SMILES: CO[C@]12C=C[C@@]3(C[C@@H]1C(C)=O)[C@H]1Cc4ccc(OCc5ccccc5)c5c4[C@@]3(CCN1Cc1ccccc1)[C@H]2O5]

3. The method according to any one of the preceding claims, wherein the one or more additives are selected from the group consisting of: a non-cyclic polyether, a cyclic polyether, a Lewis acid, and a combination thereof, for example wherein the non-cyclic polyether is a glyme.

4. The method according to claim 3, wherein the glyme is selected from the group consisting of: monoglyme, diglyme, triglyme, tetraglyme, ethyl glyme, ethyl diglyme, butyl glyme, butyl diglyme, and polyglyme, for example diglyme.

5. The method according to any one of the preceding claims, wherein the one or more additives are added to the solvent system in a stochiometry with respect to the compound Pg-O-II-Pg* of from 1 equivalent to 8 equivalents, such as from 2 equivalents to 7 equivalents, such as from 3 equivalents to 6 equivalents, for example 4 equivalents.

6. The method according to any one of the preceding claims, wherein the method comprises the steps:
a. adding the tert-butylmagnesium compound to a first solvent system to provide a solution of the tert-butylmagnesium compound;
b. adding Compound Pg-O-II-Pg* to a second solvent system to provide a solution of Pg-O-II-Pg*; and
c. adding the solution of Pg-O-II-Pg* over a time period to the solution of the tert-butylmagnesium compound; or adding the solution of the tert-butylmagnesium compound over a time period to the solution of Pg-O-II-Pg*.

7. The method according to any one of the preceding claims, wherein the time period is from 10 to 80 minutes per mL of solution of BnO-II-Bn, for example 20 minutes for 0.5 mL solution of BnO-II-Bn.

8. The method according to any one of the preceding claims, wherein BnO-II-Bn and the tert-butylmagnesium compound are mixed and allowed to react for at least 1 hour after mixing, such as from 1 hour to 24 hours after mixing.

9. The method according to any one of the preceding claims, wherein the tert-butylmagnesium compound is selected from the group consisting of: a tert-butylmagnesium halide and di-tert-butylmagnesium, for example wherein the tert-butylmagnesium compound is tert-butylmagnesium chloride.

10. The method according to any one of the preceding claims, wherein the tert-butylmagnesium compound is added to the solvent system in a stochiometry with respect to the compound BnO-II-Bn of from 2 equivalents to 12 equivalents, such as from 2 equivalents to 11 equivalents, such as from 2 equivalents to 10 equivalents, such as from 3 equivalents to 9 equivalents, such as from 4 equivalents to 8 equivalents, such as from 5 equivalents to 9 equivalents, such as from 6 to 7 equivalents, for example 6 equivalents or for example 8 equivalents.

11. The method according to any one of the preceding claims, wherein the solvent system, the first solvent system, and/or the second solvent system comprises a solvent having a dielectric constant of from 1 to 6, such as from 1 to 5.

12. The method according to any one of the preceding claims, wherein the method comprises mixing Compound BnO-II-Bn with tert-butylmagnesium chloride and diglyme in a solvent system at a temperature within the range of from 5° C. to 60° C. and allowed to react at 15° C. to 60° C. for at least 1 hour after mixing to provide Compound BnO-IIIA-Bn.

13. The method according to claim 12, wherein diglyme is added to the solvent system in a stochiometry with respect to the compound BnO-II-Bn of from 1 equivalent to 6 equivalents, such as from 2 equivalents to 6 equivalents, such as 4 equivalents.

14. The method according to any of claims 12-13, wherein the solvent system comprises a) an ethereal solvent, such as diethyl ether or cyclopentyl methyl ether, and b) a nonpolar solvent, such as toluene.

15. The method according to any of claims 12-14, wherein a) the tert-butylmagnesium chloride and diglyme is mixed in an ethereal solvent and optionally a non-polar solvent; and Compound BnO-II-Bn is added to a nonpolar solvent, such as toluene, and optionally also an ethereal solvent and then b) the compound BnO-II-Bn in the nonpolar solvent is added to the ethereal solvent of a).

16. The method according to any of claims 12-14, wherein a) the tert-butylmagnesium chloride and diglyme is mixed in cyclopentyl methyl ether (CPME) and optionally a non-polar solvent; and Compound BnO-II-Bn is added to a nonpolar solvent, such as toluene, and optionally also an ethereal solvent and then b) the compound BnO-II-Bn in the nonpolar solvent is added to the ethereal solvent of a).

17. The method according to any of claims 12-14, wherein a) the tert-butylmagnesium chloride and diglyme is mixed in cyclopentyl methyl ether (CPME) and optionally a non-polar solvent; and Compound BnO-II-Bn is added to a nonpolar solvent, such as toluene, and then b) the compound BnO-II-Bn in the nonpolar solvent is added to the CPME of a).

18. A method of preparing a compound of formula Pg-O-IIIA-Pg*, 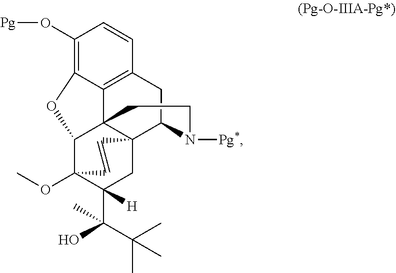
comprising mixing a compound of formula Pg-O-II-Pg*, 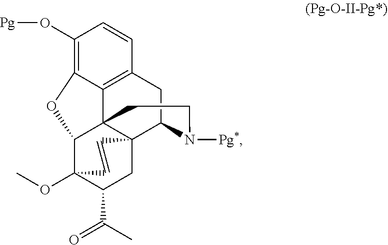
with 4 to 8 equivalents of a tert-butylmagnesium compound and 3 to 6 equivalents of diglyme in a solvent system comprising cyclopentyl methyl ether (CPME) to provide the compound of formula Pg-O-IIIA-Pg*; wherein each Pg and Pg* is a hydroxy-protecting group and Pg and Pg* the same or different; wherein each Pg and Pg* is selected from the group consisting of: benzyl (Bn), methoxymethyl (MOM), tetrahydropyranyl (THP), tert-butyl (tBu), allyl, tert-butyldimethylsilyl (TBDMS), tert-butyldiphenylsilyl (TBDPS), acetyl (Ac), pivaloyl (Piv), and benzoyl (Bz).

19. A method for isolating a compound of formula Pg-O-IIIA-Pg* from a reaction mixture in a reaction container, the method comprising:
a. adding an alcoholic solvent, such as ethanol to the reaction mixture;
b. heating the reaction mixture to at least 60° C., for example to the boiling point of the alcoholic solvent, for a predefined amount of time;
c. cooling the reaction mixture to from 40 to 55° C., optionally over the course of at least 30 minutes, forming a solid comprising the compound;
d. further cooling the reaction mixture to from 0 to 30° C. and stirring the mixture for a predefined amount of time;
e. adding further of the alcoholic solvent to the reaction mixture; and
f. collecting the solid comprising the compound by filtration and optionally drying the solid in vacuo.

20. A method for preparing buprenorphine, or a salt thereof, comprising the method as defined in any one of the preceding claims.

21. The method according to claim 20, wherein the solvent system comprises cyclopentyl methyl ether (CPME).

22. The method according to claim 20, wherein the solvent system comprises cyclopentyl methyl ether (CPME) and toluene.

23. The method according to any one of claims 20-22, wherein the method further comprises one, more or all of the steps of:
step (i)(F): in a solvent system S1 comprising a polar protic solvent, reacting Compound HO-I-H 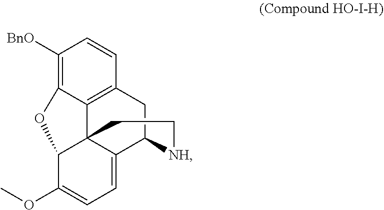 [SMILES: COC1=CC=C2[C@H]3Cc4ccc(OCc5ccccc5)c5c4[C@@]2(CCN3)[C@H]1O5]
with benzyl halide, benzyl sulfonate, or activated benzyl alcohol to provide Compound BnO-I-Bn: 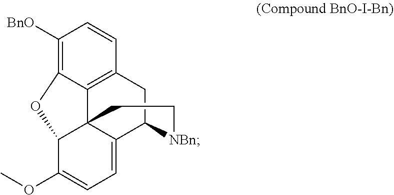 [SMILES: COC1=CC=C2[C@H]3Cc4ccc(OCc5ccccc5)c5c4[C@@]2(CCN3Cc2ccccc2)[C@H]1O5]
step (ii)(B): in a solvent system S2 comprising a polar protic solvent, reacting Compound BnO-I-Bn with methyl vinyl ketone to provide Compound BnO-II-Bn: 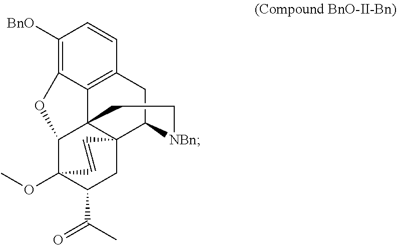 [SMILES: CO[C@]12C=C[C@@]3(C[C@@H]1C(C)=O)[C@H]1Cc4ccc(OCc5ccccc5)c5c4[C@@]3(CCN1Cc1ccccc1)[C@H]2O5]
step (iii)(D): in a solvent system S3 comprising a nonpolar solvent, reacting Compound BnO-II-Bn with a tert-butylmagnesium compound to provide Compound BnO-IIIA-Bn: 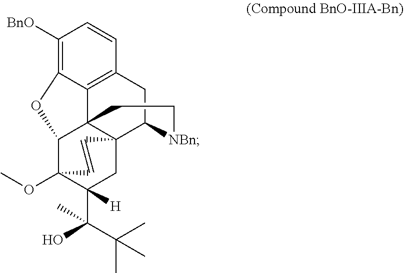 [SMILES: CO[C@]12C=C[C@@]3(C[C@@H]1[C@](C)(O)C(C)(C)C)[C@H]1Cc4ccc(OCc5ccccc5)c5c4[C@@]3(CCN1Cc1ccccc1)[C@H]2O5]
Step (iv)(C): reacting Compound BnO-IIIA-Bn with H₂ in the presence of a hydrogenation catalyst to provide a compound of Compound HO-IV-H: 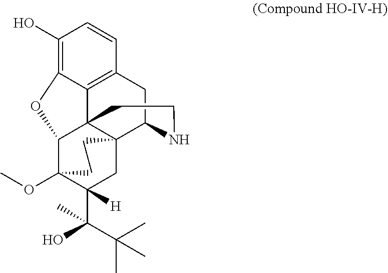 [SMILES: CO[C@]12CC[C@@]3(C[C@@H]1[C@](C)(O)C(C)(C)C)[C@H]1Cc4ccc(O)c5c4[C@@]3(CCN1)[C@H]2O5]
Step (v)(A1): reacting Compound HO-IV-H with cyclopropane carboxaldehyde followed by a hydride source; or
Step (v)(A2): reacting Compound HO-IV-H with cyclopropanecarboxylic acid halide followed by a reducing agent; or
Step (v)(A3): reacting Compound HO-IV-H with cyclopropylmethyl halide or activated cyclopropane methanol;
to provide buprenorphine.

24. The method according to any one of claims 20-23, wherein the method further comprises the method for isolating a compound of formula Pg-O-IIIA-Pg* as defined in claim 19.
